SEQUENCE LISTING (12 sequences)

SEQ ID NO: 1 - protein, 442 aa, Mus musculus
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 214 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 5 aa, Mus musculus
NYDIN

SEQ ID NO: 4 - protein, 11 aa, Mus musculus
WIFPGDGSTQY

SEQ ID NO: 5 - protein, 9 aa, Mus musculus
QTTATWFAY

SEQ ID NO: 6 - protein, 11 aa, Mus musculus
RASQSISDYLH

SEQ ID NO: 7 - protein, 7 aa, Mus musculus
YASQSIS

SEQ ID NO: 8 - protein, 9 aa, Mus musculus
QNGHSFPLT

SEQ ID NO: 9 - protein, 534 aa, Homo Sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 10 - protein, 316 aa, Homo Sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 11 - protein, 4 aa, Homo Sapiens
IRFD

SEQ ID NO: 12 - protein, 17 aa, Artificial Sequence
  8H9 Heavy Chain CDR-2 alternative
WIFPGDGSTQYNEKFKG

(19) United States
(12) Patent Application Publication
(10) Pub. No.: US 2023/0293738 A1
(43) Pub. Date: Sep. 21, 2023

(54) B7H3 Antibodies with Chelators

(71) Applicant: Y-mAbs Therapeutics, Inc., New York, NY (US)

(72) Inventors: Sonia SEQUEIRA, Verona, NJ (US); Ahmed MAHIUDDIN, Verona, NJ (US); Torben LUND-HANSEN, Roskilde (DK); Claus J. MØLLER SAN-PEDRO, Gilleleje (DK)

(21) Appl. No.: 17/917,377

(22) PCT Filed: Apr. 21, 2021
(86) PCT No.: PCT/DK2021/050118

§ 371 Date: Oct. 6, 2022

Related U.S. Application Data
(60) Provisional application No. 63/015,454, filed on Apr. 24, 2020.

(51) Int. Cl. A61K 51/10; A61P 35/00

(52) U.S. Cl. CPC A61K 51/1096; A61P 35/00; A61K 51/1045

(57) ABSTRACT

The present invention relates to B7H3-antibodies conjugated to specific chelators for radiolabeling with imaging or therapeutic radioisotopes. The invention further relates to B7H3-antibodies for treatment or theranostic use in cancer.

### FIELD OF THE INVENTION

The present invention relates to B7H3-antibodies conjugated to specific chelators for radiolabeling with imaging or therapeutic radioisotopes. The invention further relates to B7H3-antibodies conjugated to at least two chelators. The invention further relates to B7H3-antibodies for treatment or theranostic use in cancer.

### BACKGROUND

According to Modak 2001 and Xu 2009, B7H3 (also known as cluster of differentiation 276 [CD276]) is a transmembrane glycoprotein of the B7/CD28 immunoglobin superfamily that modulates immune functions in tumor surveillance, infections, and autoimmune diseases. B7H3 is an antigen overexpressed on the cell membrane of a broad spectrum of tumor types and minimally expressed in normal human tissues.

According to Kramer SIOP, 2019, Pandit-Taskar 2019 and Kramer 2017 anti-B7H3 mouse monoclonal antibody 8H9 (INN name, Omburtamab) radiolabeled with Iodine-131/Iodine-124 has been successfully used in radioimmunotherapy for patients with B7H3 positive tumors, such as neuroblastoma relapsed to the central nervous system or leptomeninges. According to Modak, CTOS, 2019 it has further been used for patients with desmoplastic small round cell tumors (DSRCT) and according to Bailey 2019 it has been used for patients with embryonal tumors with multilayered rosettes (ETMR).

According to Kramer 2019, intra-compartmental targeted radiotherapy using ¹³¹I-omburtamab has been shown to improve life expectancy in patients with neuroblastoma metastatic to the central nervous system.

Modak 2001 and Ahmed et al 2015 describe that radiolabeled omburtamab remains a promising target for radioimmunotherapy of many additional lethal cancers (Modak, 2001; Ahmed et al. 2015).

The international patent application WO2018209346A1 describes use of anti-B7H3 antibodies for treating cancer in the central nervous system (CNS). The application describes use of ¹³¹I-8H9 antibodies for the treatment of neuroblastoma and central nervous system/leptomeningeal (CNS/LM) neoplasms in adult subjects, and use of ¹²⁴I-8H9 and ¹³¹I-8H9 antibodies for the treatment of metastatic neuroblastoma, sarcoma, melanoma, ovarian carcinoma to the CNS and primary recurrent CNS malignancies including medulloblastoma/PNET, ependymoma, embryonal tumor with multi rosettes, rhabdoid tumor, chordoma and choroid plexus carcinoma.

### SUMMARY OF THE INVENTION

When labeled with a radionuclide, anti-B7H3 antibodies may target B7H3 on the cell membrane and deliver a radioactive payload to B7H3-expressing tumors, inducing DNA damage and cell death without being internalized or activating effector functions.

The main limitation of Iodine-based radiotherapies, including ¹³¹I-8H9 antibody, is that once separated from the antibody, either inter- or extracellularly, the radioiodine will redistribute to the thyroid and gastro-intestinal tract. One strategy to overcome this limitation of the ¹³¹I-8H9 antibody as a radiotherapeutic is to utilize an alternate radionuclide. Of interest is Lutetium-177, a beta-emitting radiometal, with similar half live (t1/2) to Iodine-131 (6.7 and 8 days, respectively) (Dash 2015). Lutetium 177 has a lower maximum beta emission than iodine 131 (496 and 610 keV, respectively) resulting in a shorter penetration distance (mean 0.67 mm) in soft tissue making this radionuclide ideal for delivering tumoricidal beta radiation to small volumes such as minimal residual disease following surgery, micrometastatic disease, and tumor cells near the surface of cavities, while further reducing the risk of normal tissue toxicity such as myelosuppression and negating specific toxicity to the thyroid. In addition, two photon energy peaks (ie, 208 keV and 113 keV) can be used for gamma imaging, suggesting its use as a theranostic agent. Theranostics is the term used to describe a radiopharmaceutical that can both identify (diagnose) and deliver radiotherapy to treat tumors, through a single or two different radiolabels. A straightforward radiochemistry is an additional advantage of 177Lu-labeled antibodies, reducing operator exposure. Lutetium-177 is chelated to antibodies via a chelator, such as Diethylenetriamine Pentaacetic Acid (DTPA) or Dodecane Tetraacetic Acid (DOTA). Manipulation of the chelator to antibody ratio is necessary to optimize the maximum radioactive payload while preserving immunoreactivity and stability.

This invention relates to entities such as ¹⁷⁷Lu-DTPA-8H9 antibodies, ¹⁷⁷Lu-DTPA-humanized 8H9 antibodies, ¹⁷⁷Lu-DOTA-8H9 antibodies or 177Lu-DOTA-humanized 8H9 antibodies, with different chelator to antibody (CAR) ratios. In addition, this invention relates to the use of radiolabeled DTPA- or DOTA-8H9 antibodies for the treatment and/or imaging of cancer by intracerebroventricular, intraperitoneal or intravenous administration. In particular, ¹⁷⁷Lu-DTPA-8H9 antibody CAR 3 and 3.6 are stable and bind to B7-H3 in vitro and in vivo, target tumors in vivo and show favorable dosimetry to normal organs when compared to a ¹³¹I-8H9 antibody, which is currently in clinical development. Similarly, ¹⁷⁷Lu-DOTA-8H9 antibody CAR 6.3 was well tolerated and displayed tumor targeting in animal studies.

According to Kramer et al 2017 (Abstract—A curative approach to central nervous system metastases of neuroblastoma), 108 patients with CNS neuroblastoma, were evaluated. Patients were treated with ¹³¹I-8H9 antibody administered intracerebroventricularly. At analysis, the median OS is 3.7 years [95% Cl: 1.9 to 7.5] and the 2-year OS rate is 57% [95% Cl: 47 to 67%]. The 5-year OS rate is 41% [95% Cl: 31 to 52%]. The 10-year OS rate is 37% [95% Cl: 26 to 48%]. According to Kramer et al 2017 (abstract—Safety and efficacy of intraventricular 131I-labeled monoclonal antibody 8H9 targeting the surface glycoprotein B7-H3), 57 patients with primary CNS malignancies or malignancies metastatic to the CNS received 158 injections in the outpatient setting and had favorable results, with no dose limiting toxicities. In a phase 1 clinical trial NCT04022213, 55 patients with DSRCT or other cancers of the peritoneum received ¹³¹I-8H9 antibody in combination with WA-IMRT. According to an aspect, the invention concerns antibodies or antigen binding fragments thereof conjugated to chelators, wherein the chelator-to-antibody ratio (CAR) is larger than one, and wherein said antibodies or fragments are capable of binding an antigen, wherein said antigen is B7H3.

According to another aspect, the invention concerns use of antibodies or antigen binding fragments thereof according to the invention, for the manufacture of a pharmaceutical composition, preferably for use in a treatment according to the invention.

According to another aspect, the invention concerns a pharmaceutical composition comprising antibodies or antigen binding fragments thereof according to the invention, preferably for use in a treatment of an indication according to the invention.

According to another aspect, the invention concerns a method of treatment of an indication according to the invention in a human subject comprising administration of antibodies, antigen binding fragments thereof or a pharmaceutical formulation according to the invention.

According to another aspect, the invention concerns a method of manufacturing the antibodies or antigen binding fragments thereof according to the invention, comprising the steps of:
i. Providing an antibody solution;
ii. Adding a chelator solution; and
iii. Monitoring the reaction to obtain the desired CAR range.

### DETAILED DESCRIPTION

According to an embodiment, the invention concerns antibodies or antigen binding fragments thereof conjugated to chelators, wherein the chelator-to-antibody ratio (CAR) is larger than one, and wherein said antibodies or fragments are capable of binding an antigen, wherein said antigen is B7H3.

According to an embodiment, the invention concerns the antibodies or antigen binding fragments according to the invention, wherein the chelator-to-antibody ratio (CAR) is selected among 1-10, 1.5-9, 2-8, 2.3-7, 2.4-6.5, 2.5-6.4, 6.0-6.3, 2.6-6, 3-5, 3.2-4, 3.3-3.6, and about 3.

According to an embodiment, the invention concerns the antibodies or antigen binding fragments according to the invention, wherein said chelator-to-antibody ration (CAR) is selected among 3.0, 3.6, 6.0 and 6.3.

According to an embodiment, the invention concerns the antibodies or antigen binding fragments according to the invention, wherein said chelator is selected among DOTA

(Dodecane Tetraacetic Acid), DTPA (DiethyleneTriamine Pentaacetic Acid), NOTA (Nonane Tetraacetic Acid) and DFO (Deferoxa mine).

DOTA is also referred to as 1,4,7,10-tetraazacyclododecane-1,4,7 10-tetraacetic acid, and has the formula (CH2CH2NCH2CO2H)4.

DTPA is also referred to with the IUPAC name 2-[bis[2-[bis(carboxymethyl)amino]ethyl]amino]acetic acid. DTPA has the molecular formula C14H23N3O10.

According to an embodiment, the invention concerns the antibodies or antigen binding fragments according to the invention, wherein the chelated antibodies includes DTPA, and wherein said chelator-to-antibody ratio (CAR) is 3.

The term CAR may also be used about the chelator-to-fragment ratio depending on the context.

According to an embodiment, the invention concerns the antibodies or antigen binding fragments according to the invention, wherein the chelated antibodies includes DTPA, and wherein said chelator-to-antibody ratio (CAR) is 3.6.

According to an embodiment, the invention concerns the antibodies or antigen binding fragments according to the invention, wherein the chelated antibodies includes DOTA, and wherein said chelator-to-antibody ratio (CAR) is 6.3.

According to an embodiment, the invention concerns the antibodies or antigen binding fragments according to the invention, wherein the chelated antibodies includes DOTA, and wherein said chelator-to-antibody ratio (CAR) is 3.

According to an embodiment, the invention concerns the antibodies or antigen binding fragments according to the invention, wherein the chelated antibodies includes DOTA, and wherein said chelator-to-antibody ratio (CAR) is 3.6.

According to an embodiment, the invention concerns the antibodies or antigen binding fragments according to the invention, wherein said DOTA is a variant of DOTA, such as Benzyl-DOTA.

According to an embodiment, the invention concerns the antibodies or antigen binding fragments according to the invention, wherein said DTPA is a variant of DTPA, such as CHX-A″-DTPA or p-SCN-Bn-CHX-A″-DTPA.

CHX-A″-DTPA is also referred to as N—[(R)-2-Amino-3-(p-aminophenyl)propyl]-trans-(S,S)-cyclohexane-1,2-diamine-N,N,N,N,N-pentaacetic acid p-SCN-Bn-CHX-A″-DTPA is also referred to as [(R)-2-Amino-3-(4-isothiocyanatophenyl)propyl]-trans-(S,S)-cyclohexane-1,2-diamine-pentaacetic acid and has the chemical formula C₂₆H₃₄N₄O₁₀·3HCl

According to an embodiment, the invention concerns the antibodies or antigen binding fragments according to the invention, wherein said chelator compound is bound to a radioactive isotope.

According to an embodiment, the invention concerns the antibodies or antigen binding fragments according to the invention, wherein said radioactive isotope is selected among a PET label and a SPECT label.

PET may also be referred to as Positron Emission Tomography. SPECT may also be referred to as Photon Emission Computed Tomography.

According to an embodiment, the invention concerns the antibodies or antigen binding fragments according to the invention, wherein said PET label is selected among ¹²⁴I, ¹⁸F, ⁶⁴Cu and ⁸⁹Zr.

According to an embodiment, the invention concerns the antibodies or antigen binding fragments according to the invention, wherein said SPECT label is selected among ¹³¹I, ¹⁷⁷Lu, ⁹⁹mTc and ⁸⁹Zr.

According to an embodiment, the invention concerns the antibodies or antigen binding fragments according to the invention, wherein said radioactive isotope is an alpha, beta or positron emitting radionuclide.

According to an embodiment, the invention concerns the antibodies or antigen binding fragments according to the invention, wherein said radioactive isotope is selected from the group consisting of ¹²⁴I, ¹³¹I, ¹⁷⁷Lu, ⁹⁹mTc, ¹⁸F, ⁶⁴Cu and ⁸⁹Zr.

According to an embodiment, the invention concerns the antibodies or antigen binding fragments according to the invention, wherein said antibodies or antigen binding fragments comprise a structure selected among IgG, IgG1, IgG2, IgG3, and IgG4.

According to an embodiment, the invention concerns the antibodies or antigen binding fragments according to the invention, wherein said antibodies or antigen binding fragments comprise a structure selected among IgG, IgM, IgA, IgD, and IgE.

According to an embodiment, the invention concerns the antibodies or antigen binding fragments according to the invention, wherein said antibodies or antigen binding fragments comprise a Fc region which does not interact with a Fc gamma receptor.

According to an embodiment, the invention concerns the antibodies or antigen binding fragments according to the invention, wherein said antibodies or antigen binding fragments further comprises an Fc region, wherein said Fc region is not reactive or exhibit little reactivity.

According to an embodiment, the invention concerns the antibodies or antigen binding fragments according to the invention, wherein said antibodies or fragments are for use in a method of treatment of a disease.

According to an embodiment, the invention concerns the antibodies or antigen binding fragments according to the invention, wherein said disease is a cancer.

According to an embodiment, the invention concerns the antibodies or antigen binding fragments according to the invention, wherein said cancer is a metastasis.

According to an embodiment, the invention concerns the antibodies or antigen binding fragments according to the invention, wherein said cancer and/or metastasis is prostate cancer, a desmoplastic small round cell tumor, ovarian cancer, gastric cancer, pancreatic cancer, liver cancer, renal cancer, breast cancer, non-small cell lung cancer, melanoma, alveolar rhabdomyosarcoma, embryonal rhabdomyosarcoma, Ewing sarcoma, Wilms tumor, neuroblastoma, ganglioneuroblastoma, ganglioneuroma, medulloblastoma, high-grade glioma, diffuse intrinsic pontine glioma, embryonal tumors with multilayered rosettes, or a cancer expressing B7H3.

According to an embodiment, the invention concerns the antibodies or antigen binding fragments thereof according to the invention, wherein said cancer is metastatic to leptomeninges.

According to an embodiment, the invention concerns the antibodies or antigen binding fragments thereof according to the invention, wherein said antibodies or antigen binding fragments are murine antibodies or antigen binding fragments thereof.

According to an embodiment, the invention concerns the antibodies or antigen binding fragments thereof according to the invention, wherein said antibodies or antigen binding fragments are humanized antibodies or antigen binding fragments thereof.

According to an embodiment, the invention concerns the antibodies or antigen binding fragments thereof according to the invention, wherein said antibodies or antigen binding fragments thereof are chimeric antibodies or antigen binding fragments thereof.

According to an embodiment, the invention concerns the antibodies or antigen binding fragments thereof according to the invention, wherein said antibodies or antigen binding fragments are human antibodies and antigen binding fragments thereof.

According to an embodiment, the invention concerns the antibodies or antigen binding fragments thereof according to the invention, wherein said antibodies or antigen binding fragments binds to FG-loop of B7H3.

According to an embodiment, the invention concerns the antibodies or antigen binding fragments thereof according to the invention, wherein said antibodies or antigen binding fragments comprise a heavy chain sequence according to SEQ ID No.: 1 and/or a light chain sequence according to SEQ ID No.: 2

According to an embodiment, the invention concerns the antibodies or antigen binding fragments thereof according to the invention, wherein said antibodies or antigen binding fragments comprise a heavy chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No.: 1 and/or a light chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No.: 2.

According to an embodiment, the invention concerns the antibodies or antigen binding fragments thereof according to the invention, wherein said antibodies or antigen binding fragments comprise at least one sequence selected among a heavy chain variable region CDR1 according to SEQ ID No.: 3, a heavy chain variable region CDR2 according to SEQ IN No.: 4, a heavy chain variable region CDR3 according to SEQ IN No.: 5 a light chain variable region CDR1 according to SEQ ID No.: 6, a light chain variable region CDR2 according to SEQ ID No.: 7 and a light chain variable region CDR3 according to SEQ ID No.: 8.

Alternatively, the heavy chain variable region CDR2 might be defined as comprising a sequence according to SEQ IN No.: 12.

According to an embodiment, the invention concerns the antibodies or antigen binding fragments according to the invention, wherein said antibodies or antigen binding fragments bind to an antigen, wherein said antigen is B7H3.

According to an embodiment, the invention concerns the antibodies or antigen binding fragments according to the invention, wherein said antibodies or antigen binding fragments bind to an epitope, and wherein said epitope is an epitope of B7H3.

According to an embodiment, the invention concerns the antibodies or antigen binding fragments according to the invention, wherein said antibodies or antigen binding fragments bind to the sequence according to SEQ ID No.: 9, 10 and 11.

According to an embodiment, the invention concerns the antibodies or antigen binding fragments according to the invention, wherein said antibodies or antigen binding fragments are administered intrathecally to a subject.

According to an embodiment, the invention concerns the antibodies or antigen binding fragments according to the invention, wherein said antibodies or antigen binding fragments are administered to the subject via an intraventricular device.

According to an embodiment, the invention concerns the antibodies or antigen binding fragments according to the invention, wherein said intraventricular device is an intraventricular catheter.

According to an embodiment, the invention concerns the antibodies or antigen binding fragments according to the invention, wherein said intraventricular device is an intraventricular reservoir.

According to an embodiment, the invention concerns the antibodies or antigen binding fragments according to the invention, wherein said antibodies or antigen binding fragments are for treatment of a human being.

According to an embodiment, the invention concerns the antibodies or antigen binding fragments according to the invention, wherein said human being is under 18 years old.

According to an embodiment, the invention concerns the antibodies or antigen binding fragments according to the invention, wherein said human is at least 18 years old.

According to an embodiment, the invention concerns use of an antibodies or antigen binding fragments thereof according to the invention, for the manufacture of a pharmaceutical composition, preferably for use in a treatment according to the invention.

According to an embodiment, the invention concerns a pharmaceutical composition comprising antibodies or antigen binding fragments thereof according to the invention, preferably for use in a treatment of an indication according to the invention.

According to an embodiment, the invention concerns a method of treatment of an indication according to the invention in a human subject comprising administration of antibodies, antigen binding fragments thereof or a pharmaceutical formulation according to the invention.

According to an embodiment, the invention concerns the method, comprising administering to the subject one treatment cycle of the antibodies, antigen-binding fragments thereof or composition.

According to an embodiment, the invention concerns the method, comprising administering to the subject two treatment cycles of the antibodies or antigen-binding fragments thereof.

According to an embodiment, the invention concerns the method, wherein one treatment cycle comprises a dosimetry dose and a treatment dose.

According to an embodiment, the invention concerns the method, wherein the therapeutically effective amount is from about 10 mCi to about 200 mCi or from about 10mCi to about 100 mCi.

According to an embodiment, the invention concerns the method, wherein the therapeutically effective amount is about 50 mCi.

According to an embodiment, the invention concerns the method, wherein the method prolongs survival of the subject.

According to an embodiment, the invention concerns the method, wherein the method prolongs remission of the cancer in the subject.

According to an embodiment the invention concerns a method of manufacturing the antibodies or antigen binding fragments thereof according to the invention, comprising the steps of:
i. Providing an antibody solution;
ii. Adding a chelator solution; and
iii. Monitor the reaction to obtain the desired CAR range.

According to an embodiment the invention concerns the method of manufacturing further comprising a step of subjecting said antibody solution to Tangential Flow Filtration (TFF) and exchanging with a buffer before adding said chelator solution.

According to an embodiment the invention concerns the method of manufacturing wherein the antibodies or antigen binding fragments thereof are for use in a method of treatment according to the invention.

According to an embodiment the invention concerns the method of manufacturing comprising a step of random lysine conjugation process.

The term random lysine conjugation refers to a conventional conjugation strategy involving random conjugation to lysine amines on cysteines of the antibody, which is a common method to produce antibody conjugates and is suitable for most in vitro applications.

According to an embodiment the invention concerns the method of manufacturing further comprising a step of: Filtering to remove any precipitate formed, optionally after other process steps mentioned above.

According to an embodiment the invention concerns the method of manufacturing further comprising a step of size exclusion chromatography (SEC) to determine the concentration of conjugate in solution.

According to an embodiment the invention concerns the method of manufacturing further comprising a step of adding a poloxamer.

According to an embodiment the invention concerns the method of manufacturing further comprising a step of adding a buffer.

According to an embodiment the invention concerns the method of manufacturing wherein the final yield of antibodies or antigen binding fragments thereof is at least 50%, at least 55%, at least 60%, at least 65%, at least 70%, at least 75%, at least 80%, at least 85%, at least 90%, at least 95% or at least 99%.

According to an embodiment the invention concerns the method of manufacturing wherein the antibodies or antigen binding fragments has a chelator-to-antibody ratio (CAR) selected among 1.1-10, 1.5-9, 2-8, 2.3-7, 2.4-6.5, 2.5-6.4, 6.0-6.3, 2.6-6, 3-5, 3.2-4, 3.3-3.6.

In order to provide a clear and consistent understanding of the specification and claims, including the scope to be given such terms, the following definitions are provided.

Affinity: As is known in the art, “affinity” is a measure of the tightness with which a particular ligand (e.g., an antibody) binds to its partner (e.g., an epitope). Affinities can be measured in different ways.

Antibody: The term “antibody” is art-recognized terminology and is intended to include molecules or active fragments of molecules that bind to known antigens. Examples of active fragments of molecules that bind to known antigens include Fab and F(ab′)2 fragments. These active fragments can be derived from an antibody of the present invention by a number of techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. The term “antibody” also includes bispecific and chimeric antibodies and other available formats.

Antibody fragment: An antibody fragment is a portion of an antibody such as F(ab′)2, F(ab)2, Fab′, Fab, Fv, sFv and the like. Regardless of structure, an antibody fragment binds with the same antigen that is recognized by the intact antibody. For example, an 3F8 monoclonal antibody fragment binds with an epitope recognized by 3F8. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

B7H3 and B7-H3 are used interchangeable to refer to the same antigen.

Bispecific antibody: A bispecific antibody is an antibody that can bind simultaneously to two targets which are of different structure. Bispecific antibodies (bsAb) and bispecific antibody fragments (bsFab) have at least one arm that specifically binds to an antigen, for example, GD2 and at least one other arm that specifically binds to another antigen, for example a targetable conjugate that bears a therapeutic or diagnostic agent. A variety of bispecific fusion proteins can be produced using molecular engineering. In one form, the bispecific fusion protein is divalent, consisting of, for example, a scFv with a single binding site for one antigen and a Fab fragment with a single binding site for a second antigen. In another form, the bispecific fusion protein is tetravalent, consisting of, for example, an IgG with two binding sites for one antigen and two identical scFv for a second antigen.

Chimeric antibody: A chimeric antibody is a recombinant protein that contains the variable domains including the complementarity-determining regions (CDRs) of an antibody derived from one species, for example a rodent antibody, while the constant domains of the antibody molecule is derived from those of a human antibody. The constant domains of the chimeric antibody may also be derived from that of other species, such as a cat or dog.

Effective amount: As used herein, the term “effective amount” refers to an amount of a given compound, conjugate or composition that is necessary or sufficient to realize a desired biologic effect. An effective amount of a given compound, conjugate or composition in accordance with the methods of the present invention would be the amount that achieves this selected result, and such an amount can be determined as a matter of routine by a person skilled in the art, without the need for undue experimentation.

Humanized antibody: A humanized antibody is a recombinant protein in which the CDRs from an antibody from one species; e.g., a rodent antibody, is transferred from the heavy and light variable chains of the rodent antibody into human heavy and light variable domains. The constant domain of the antibody molecule is derived from those of a human antibody.

A human antibody may be an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas.

Prevent: As used herein, the terms “prevent”, “preventing” and “prevention” refer to the prevention of the recurrence or onset of one or more symptoms of a disorder in a subject as result of the administration of a prophylactic or therapeutic agent.

Radioactive isotope: Examples of radioactive isotopes that can be conjugated to antibodies for use diagnostically or therapeutically include, but are not limited to, ²¹¹At, ¹⁴C, ⁵¹Cr, ⁵⁷CO, ⁵⁸CO, ⁶⁷CU, ¹⁵²EU, ⁶⁷Ga, ³H, ¹¹¹In, ⁵⁹Fe, ²¹²Pb, ¹⁷⁷Lu, ³²P, ²²³Ra, ²²⁴Ra, ¹⁸⁶Re, ¹⁸⁸Re, ⁷⁵Se, ³⁵S, ^{99m}Tc, ²²⁷Th, ⁸⁹Zr, ⁹⁰Y, ¹²³I, ¹²⁴I, ¹²⁵I, ¹³¹I, ^{94m}Tc, ⁶⁴cu, ⁶⁸Ga, ⁶⁶Ga, ⁷⁶Br, ⁸⁶Y, ⁸²Rb, ^{110m}In, ¹³N, ¹¹C, ¹⁸F and alpha-emitting particles. Non-limiting examples of alpha-emitting particles include ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.

Subject: By “subject” or “individual” or “animal” or “patient” or “mammal,” is meant any subject, particularly a mammalian subject, for whom diagnosis, prognosis, or therapy is desired. Mammalian subjects include humans and other primates, domestic animals, farm animals, and zoo, sports, or pet animals such as dogs, cats, guinea pigs, rabbits, rats, mice, horses, cattle, cows, and the like.

Treatment: As used herein, the terms “treatment”, “treat”, “treated” or “treating” refer to prophylaxis and/or therapy, particularly wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### FIGURES

FIG. 1 shows effect of CHX-A″-DTPA Conjugation Ratio and the Lutetium-175 Label on 8H9 antibody comprising a light chain according to SEQ ID No.: 2 and heavy chain according to SEQ ID No.: 1. Affinity is binding to Human 41g-B7H3.

FIG. 2A and FIG. 2B shows in vivo % ID/g of tissue after intravenous injection of ¹⁷⁷Lu-DTPA-8H9 antibody and ¹²⁵I-8H9 antibody in animals bearing DAOY medulloblastoma xenografted tumors. ID/g=injected dose per gram; DTPA=p-SCN-Bn-CHX-A″-DTPA. Note: Data are presented as mean±standard error of the mean (left panel) and mean with individual data (right panel).

FIG. 3 shows schematically a procedure for manufacturing a conjugate between bifunctional chelating agents and 8H9 antibodies.

FIG. 4 shows schematically a procedure for manufacturing a conjugate between bifunctional chelating agents and 8H9 antibodies.

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Example 1

¹⁷⁷Lu-DPTA-8H9 Antibody Comprising a Light Chain According to SEQ ID No.: 2 and Heavy Chain According to SEQ ID No.: 1 (CAR3) and ¹⁷⁷Lu-DOTA-8H9 Antibody Comprising a Light Chain According to SEQ ID No.: 2 and Heavy Chain According to SEQ ID No.: 1 (CAR6.3) Radiolabeling Overview

A brief overview is provided below.
1. If needed, the DTPA-8H9 antibody comprising a light chain according to SEQ ID No.: 2 and heavy chain according to SEQ ID No.: 1 or DOTA-8H9 antibody comprising a light chain according to SEQ ID No.: 2 and heavy chain according to SEQ ID No.: 1 derivatives were buffer exchanged prior to use in the reaction.
   i. The required amount of 8H9 antibody comprising a light chain according to SEQ ID No.: 2 and heavy chain according to SEQ ID No.: 1 derivative solution was transferred (0.5 mg-1.0 mg) into an ultrafiltration tube (a 50 kDa Amicon Ultra Filter, Millipore Ref #UFC95024, or equivalent).
   ii. The 8H9 antibody comprising a light chain according to SEQ ID No.: 2 and heavy chain according to SEQ ID No.: 1 derivative was diluted (3 mL) with HEPES buffer (0.1 M, pH 5.5).
   iii. The tube was centrifuged (4000 rpm, 10 min) to reduce the 8H9 antibody comprising a light chain according to SEQ ID No.: 2 and heavy chain according to SEQ ID No.: 1 derivative solution volume by a factor of 2-3.
   iv. The ultrafiltrate was discarded and then steps (ii) and (iii) were repeated at least three times.
   v. During the last centrifuge, when the volume was reduced to a level that corresponded to an 8H9 antibody comprising a light chain according to SEQ ID No.: 2 and heavy chain according to SEQ ID No.: 1 derivative concentration of ^{˜}5 mg/mL, the pH was checked (target pH 5.5) and the final contents of the ultrafiltration tube were transferred to a metal-free plastic test tube.
2. At the beginning of each reaction the appropriate labeling buffer (0.3 mL) was added to the reaction vial. When the ¹⁷⁷LuCl₃ was delivered in 0.04N HCl solution, HEPES buffer (0.1 M, pH 5.7) was used; otherwise, either MES buffer (0.5 M, pH 5.5) or HEPES buffer (0.5 M, pH 5.5) was used.
3. The 8H9 antibody comprising a light chain according to SEQ ID No.: 2 and heavy chain according to SEQ ID No.: 1-derivative (approximately 50-150 μg) was then added to the reaction vial containing the required amount of buffer solution and gently mixed by flicking the vial.
4. Approximately 5-15 mCi Lutetium-177 (15-25 μL of ¹⁷⁷LuCl₃ solution) was added to the reaction vial.
5. The reaction vial was placed in a heating block set at 38° C. and the reaction was monitored by iTLC per the procedure listed below after 1 hr.
   i. A 5 μL sample was removed from the reaction vial and 3 μL of that sample was spotted on a Biodex TLC strip.
   ii. The strip was developed by placing the strip in a solution with ammonium acetate buffer (0.1M, containing 5 mM EDTA).
   iii. The labeled DOTA/DTPA-8H9 antibody comprising a light chain according to SEQ ID No.: 2 and heavy chain according to SEQ ID No.: 1 remained close to the baseline and had a Rf (Retention factor) of −0.1 while free Lu-177 travelled with the solvent front and had a Rf>0.5; acceptance criteria: RCP>95%
6. Once the reaction was determined complete via iTLC (RCP>95%), an H PLC-SEC analysis was performed.
7. If needed, the material was purified using an Amicon spin column, 30 kDa cut-off (2 mL microcentrifuge tubes). Specifically, the column was first preconditioned with labeling buffer or 1% HSA in PBS. The crude reaction material was then diluted to approximately 0.5 mL with additional labeling buffer and concentrated by spinning at 10000 rpm for 5 minutes at which time the volume was reduced from 0.5 mL to approximately 0.05 mL. This process was repeated at least four additional times with 1×PBS and the product was isolated in approximately 0.2 mL of PBS.
8. The purified product was diluted with 5% HSA in PBS as needed.

### Example 2

8H9 Antibody Comprising a Light Chain According to SEQ ID No.: 2 and Heavy Chain According to SEQ ID No.: 1.

Cross-Reactivity in Normal Human and Cynomolgus Monkey Tissue

The potential of 8H9 antibody comprising a light chain according to SEQ ID No.: 2 and heavy chain according to SEQ ID No.: 1 to bind to unintended targets was evaluated in histologically normal tissues of human or monkey origin analyzed for reactivity with 8H9 antibody comprising a light chain according to SEQ ID No.: 2 and heavy chain according to SEQ ID No.: 1 (2 μg/mL) by immunohistochemistry (IHC) (Modak 2001). A nonspecific mouse IgG1 was used as a negative control. Tissues evaluated and the reactivity of 8H9 antibody IgG1 mAb with normal tissue are shown in Table 1.

TABLE 1
8H9 antibody comprising a light chain according to SEQ
ID No.: 2 and heavy chain according to SEQ ID No.: 1.
Reactivity in Normal Human and Cynomolgus Monkey Tissues

| | |
|---|---|
| | 8H9 antibody comprising a light chain |
| | according to SEQ ID No.: 2 and heavy chain |
| Tissue Type | according to SEQ ID No.: 1 Reactivity |

Human Tissues

| | |
|---|---|
| Frontal lobe | Negative |
| Pons | Negative |
| Spinal cord | Negative |
| Cerebellum | Negative |
| Lung | Negative |
| Heart | Negative |
| Skeletal muscle | Negative |
| Thyroid | Negative |
| Testes | Negative |
| Pancreas | Cytoplasmic staining |
| Adrenal cortex | Cytoplasmic staining |
| Liver | Cytoplasmic staining |
| Sigmoid colon | Negative |
| Bone marrow | Negative |
| Kidney | Negative |

Cynomolgus Monkey Tissues

| | |
|---|---|
| Cerebellum | Negative |
| Frontal lobe | Negative |
| Occipital cortex | Negative |
| Brainstem | Negative |
| Liver | Cytoplasmic staining |
| Stomach | Negative |
| Adrenal cortex | Cytoplasmic staining |
| Kidney | Negative |

Normal human tissues were mostly negative for 8H9 antibody immunoreactivity, with the exception of pancreas, adrenal cortex, and liver, where heterogenous cytoplasmic staining was detected. Immunostaining was absent in normal human brain and bone marrow tissue sections. A similar immunoreactivity profile was observed in normal tissues from monkey. Normal monkey brain sections were negative for 8H9 antibody immunostaining. The liver and adrenal cortex displayed heterogenous cytoplasmic staining. The results suggested non-cancerous human and monkey tissues do not express, or minimally express, membrane bound 8H9 antibody antigen.

### Example 3

Binding of 8H9 Antibody Comprising a Light Chain According to SEQ ID No.: 2 and Heavy Chain According to SEQ ID No.: 1 to B7-H3 of Different Species

The binding affinity of 8H9 antibody comprising a light chain according to SEQ ID No.: 2 and heavy chain according to SEQ ID No.: 1 for recombinant B7-H3 antigens (3 μg/mL) from mouse, rat, monkey, and human was determined using surface plasmon resonance (SPR). All measurements were done in triplicate. The 8H9 antibody bound to monkey and human B7-H3 with high affinity (Table). There was no detectable binding for mouse or rat B7-H3.

TABLE 2
Binding Kinetics of 8H9 antibody comprising a light
chain according to SEQ ID No.: 2 and heavy chain according
to SEQ ID No.: 1to B7-H3 of Different Species

| | kₐ | k_{d} | K_{D} | Rₘₐₓ |
|---|---|---|---|---|
| Species | (1/M s) | (1/s) | (pM) | (RU) |
| Mouse | NA | NA | NA | 5.2 |
| Rat | NA | NA | NA | 2.2 |
| Monkey | 6.5 × 10⁶ | 1.0 × 10⁻⁵ | 1.6 | 210 |
| Human | 8.9 × 10⁶ | 1.0 × 10⁻⁵ | 1.1 | 562 |

kₐ = association constant; k_{d} = dissociation constant; K_{D} = equilibrium dissociation constant; NA = not applicable; Rₘₐₓ = maximum binding.

### Example 4

8H9-Antibody Comprising a Light Chain According to SEQ ID No.: 2 and Heavy Chain According to SEQ ID No.: 1 Binding to Recombinant Human B7H3 after Conjugation with p-SCN-Bn-DOTA or p-SCN-Bn-CHX-A″-DTPA Moieties

8H9-antibody comprising a light chain according to SEQ ID No.: 2 and heavy chain according to SEQ ID No.: 1 samples that were conjugated to the bifunctional chelators p-SCN-Bn-CHX-A″-DTPA (CAS 157380-45-5) or p-SCN-Bn-DOTA (CAS 127985-74-4) and labelled with cold (non-radioactive) lutetium were tested for the ability to bind recombinant human B7H3 protein by Surface Plasmon Resonance (SPR) and compared to the parent 8H9 antibody comprising a light chain according to SEQ ID No.: 2 and heavy chain according to SEQ ID No.: 1.

Analyses of binding to B7H3 were performed using a Biacore T200 biosensor (Biacore AB of GE Healthcare, Uppsala, Sweden).

Both human B7H3 41g and 21g proteins were dissolved in PBS (Phosphate-Buffered Saline) to make 0.1 mg/ml stock solution and stored in −80° C. B7-H3 proteins were immobilized onto the CM5 sensor chip using Amine Coupling Kit. Both proteins were diluted to 10 ug/ml with 10 mM Sodium acetate, pH 5.0. B7H3-41g-His was immobilized at 1000 RU and B7H3-21g-His at 500 RU onto active surface using Immobilization Wizard in the Biacore T200 Control Software. A blank immobilized surface was used as a control

Binding Assays:
1. Antibodies were diluted in HBS-EP buffer (10 mM HEPES, 150 mM NaCl, 3 mM EDTA, 0.05% Surfactant P20, pH 7.4) at varying concentrations (25-50-100-200-400 nM) prior to analysis.
2. Samples (60 ul) were injected over the sensor surface at a flow rate of 30 ul/min over 2 min.
3. Following completion of the association phase, dissociation was monitored in HBS-EP buffer for 10 min at the same flow rate.
4. At the end of each cycle, the surface was regenerated using 10 mM NaOH at a flow rate of 50 ul/min over 2×15 sec.

Kinetic Analysis of Biosensor Data:

The biosensor curves obtained following injection of the samples over the active surface were subtracted with the control curves obtained with the samples injected over the reference surface prior to kinetics analysis. The data were analyzed by the 1:1 fitting model and default parameter setting for the rate constants using the Biacore T200 Evaluation Software, and the apparent association on rate constant (kon, ka), dissociation off rate constant (koff, kd) and equilibrium dissociation constant (KD=kd/ka) were calculated.

To assess the effect of conjugation with p-SCN-Bn-CHX-A″-DTPA or p-SCN-Bn-DOTA on antibody affinity, the 8H9-antibody comprising a light chain according to SEQ ID No.: 2 and heavy chain according to SEQ ID No.: 1 was conjugated with different ratios of conjugate/antibody (CAR: conjugate/antibody ratio).

Normalized SPR sensorgrams of 8H9-antibody comprising a light chain according to SEQ ID No.: 2 and heavy chain according to SEQ ID No.: 1] conjugates at 400 nM concentration binding to human 21g-B7H3 were obtained, and the extrapolated kinetic data presented in Table 3.

TABLE 3
8H9-antibody comprising a light chain according to SEQ ID No.: 2 and
heavy chain according to SEQ ID No.: 1 binding kinetics to human 2Ig-
B7H3 after conjugation using p-SCN-Bn-CHX-A″-DTPA or p-SCN-Bn-DOTA

| | Ka | Kd | K_{D} | T ½ |
|---|---|---|---|---|
| Sample | (1/Ms) | (1/s) | (M) | (s) |
| m8H9(PHB800) | 4.34E+04 | 2.38E−07* | 5.50E−12 | 2.91E+06 |
| m8H9(8H9 antibody) P76501A | 4.62E+04 | 5.68E−07* | 1.23E−11 | 1.22E+06 |
| m8H9(S219) | 4.09E+04 | 1.70E−08* | 4.16E−13 | 4.07E+07 |
| Ref1-CHX-A″-DTPA (CAR 1.4) | 2.52E+04 | 4.88E−05 | 1.93E−09 | 1.42E+04 |
| Ref2-CHX-A″-DTPA (CAR 3.6) | 1.62E+04 | 1.54E−04 | 9.46E−09 | 4.51E+03 |
| Ref3-CHX-A″-DTPA (CAR 6.1) | 2.01E+04 | 4.89E−04 | 2.43E−08 | 1.42E+03 |
| Ref4-DOTA (CAR 2.6) | 2.37E+04 | 1.37E−05 | 5.80E−10 | 5.04E+04 |
| Ref5-DOTA (CAR 7.5) | 1.71E+04 | 2.93E−05 | 1.71E−09 | 2.37E+04 |
| Ref6-DOTA (CAR 11.7) | 1.73E+04 | 5.17E−05 | 2.99E−09 | 1.34E+04 |
| Ref7-DOTA (CAR 0.8) | 2.96E+04 | 4.93E−06* | 1.66E−10 | 1.41E+05 |
| Ref8-DOTA (CAR 2.4) | 2.11E+04 | 4.69E−06* | 2.22E−10 | 1.48E+05 |
| Ref conj-CHX-A-DTPA (CAR 0.6) | 2.75E+04 | 3.36E−05 | 1.22E−09 | 2.06E+04 |

CAR = chelator-to-antibody ratio; DTPA = p-SCN-Bn-CHX-A″-DTPA; DOTA = p-SCN-Bn-DOTA; kₐ = association constant; k_{d} = dissociation constant; K_{D} = equilibrium dissociation constant; T½ = half-life.
*kd below 1e−05 is beyond the fitting limits of the Biacore T200. K_{D} was calculated as kd/ka

Normalized SPR sensorgrams of 8H9-antibody comprising a light chain according to SEQ ID No.: 2 and heavy chain according to SEQ ID No.: 1 conjugates at 400 nM concentration binding to human 41g-B7H3 were obtained, and the extrapolated kinetic data presented in Table 4.

TABLE 4
8H9-antibody comprising a light chain according to SEQ ID No.: 2 and
heavy chain according to SEQ ID No.: 1 binding kinetics to 4Ig-B7H3
after conjugation using p-SCN-Bn-CHX-A″-DTPA or p-SCN-Bn-DOTA.

| | ka | kd | KD | t ½ |
|---|---|---|---|---|
| Sample | (1/Ms) | (1/s) | (M) | (s) |
| m8H9(PHB800) | 2.52E+04 | 1.02E−04 | 4.07E−09 | 6.77E+03 |
| m8H9(8H9 antibody) P76501A | 2.67E+04 | 1.01E−04 | 3.78E−09 | 6.87E+03 |
| m8H9(S219) | 2.57E+04 | 9.48E−05 | 3.69E−09 | 7.31E+03 |
| Ref1-CHX-A″-DTPA (CAR 1.4) | 1.77E+04 | 1.77E−04 | 1.00E−08 | 3.91E+03 |
| Ref2-CHX-A″-DTPA (CAR 3.6) | 1.41E+04 | 3.79E−04 | 2.69E−08 | 1.83E+03 |
| Ref3-CHX-A″-DTPA (CAR 6.1) | 1.66E+04 | 8.96E−04 | 5.38E−08 | 7.74E+02 |
| Ref4-DOTA (CAR 2.6) | 1.79E+04 | 1.36E−04 | 7.57E−09 | 5.11E+03 |
| Ref5-DOTA (CAR 7.5) | 1.28E+04 | 1.86E−04 | 1.46E−08 | 3.72E+03 |
| Ref6-DOTA (CAR 11.7) | 1.35E+04 | 2.55E−04 | 1.89E−08 | 2.72E+03 |
| Ref7-DOTA (CAR 0.8) | 2.00E+04 | 1.15E−04 | 5.76E−09 | 6.02E+03 |
| Ref8-DOTA (CAR 2.4) | 1.59E+04 | 1.37E−04 | 8.61E−09 | 5.05E+03 |
| Ref conj-CHX-A-DTPA (CAR 0.6) | 2.16E+04 | 2.18E−04 | 1.01E−08 | 3.18E+03 |

CAR = chelator-to-antibody ratio;
DTPA = p-SCN-Bn-CHX-A″-DTPA;
DOTA = p-SCN-Bn-DOTA;
kₐ = association constant;
k_{d} = dissociation constant;
K_{D} = equilibrium dissociation constant;
T½ = half-life

SPR Measurements of Lutetium Labeled 8H9-Antibody Comprising a Light Chain According to SEQ ID No.: 2 and Heavy Chain According to SEQ ID No.: 1 Conjugates

8H9-antibody comprising a light chain according to SEQ ID No.: 2 and heavy chain according to SEQ ID No.: 1 conjugates were labeled with cold Lutetium-175 and then measured for their binding to human 21g- or 41g-B7H3. Samples were compared to unlabeled 8H9-antibody comprising a light chain according to SEQ ID No.: 2 and heavy chain according to SEQ ID No.: 1, or 8H9-antibody comprising a light chain according to SEQ ID No.: 2 and heavy chain according to SEQ ID No.: 1 labeled with 1271. Unlabeled and 1271-labeled humanized 8H9-antibody comprising a light chain according to SEQ ID No.: 2 and heavy chain according to SEQ ID No.: 1 were also included in the analysis. Data is shown in Tables 5 and 6.

TABLE 5
Effect of 8H9-antibody comprising a light chain according
to SEQ ID No.: 2 and heavy chain according to SEQ ID No.:
1 conjugation and labeling on affinity to 2Ig-B7H3.

| | ka | kd | KD | t ½ |
|---|---|---|---|---|
| Sample | (1/Ms) | (1/s) | (M) | (s) |
| 175Lu-DTPA-8H9-antibody (CAR1.4) | 3.16E+04 | 6.87E−06* | 2.17E−10 | 1.01E+05 |
| 175Lu-DTPA-8H9-antibody (CAR3) | 1.80E+04 | 3.23E−05 | 1.80E−09 | 2.14E+04 |
| 175Lu-DTPA-8H9-antibody (CAR3.6) | 1.65E+04 | 3.86E−05 | 2.34E−09 | 1.80E+04 |
| 175Lu-DTPA-8H9-antibody (CAR6.1) | 1.46E+04 | 1.06E−04 | 7.21E−09 | 6.57E+03 |
| 175Lu-DOTA-8H9-antibody (CAR2.6) | 1.81E+04 | 3.16E−05 | 1.74E−09 | 2.20E+04 |
| 175Lu-DOTA-8H9-antibody (CAR6.3) | 1.52E+04 | 3.57E−05 | 2.34E−09 | 1.94E+04 |
| 127I-Humanized-8H9-antibody | 3.61E+05 | 7.66E−07* | 2.12E−12 | 9.04E+05 |
| 127I-8H9-antibody P76501A | 1.27E+05 | 6.24E−07* | 4.91E−12 | 1.11E+06 |
| YMS1(hu8H9/Humanized 8H9- | 1.87E+05 | 1.78E−07* | 9.54E−13 | 3.88E+06 |
| antibody) | | | | |
| hu8H9-3.1 | 4.59E+04 | 1.02E−04 | 2.22E−09 | 6.81E+03 |
| 8H9 (8H9-antibody) P76501A | 5.38E+04 | 1.10E−07* | 2.05E−12 | 6.30E+06 |
| 8H9 (S219) | 4.71E+04 | 4.74E−08* | 1.01E−12 | 1.46E+07 |

CAR = chelator-to-antibody ratio;
DTPA = p-SCN-Bn-CHX-A″-DTPA;
DOTA = p-SCN-Bn-DOTA;
kₐ = association constant;
k_{d} = dissociation constant;
K_{D} = equilibrium dissociation constant;
T½ = half-life
*kd below 1e−05 is beyond the fitting limits of the Biacore T200.

TABLE 6
Effect of 8H9-antibody comprising a light chain according
to SEQ ID No.: 2 and heavy chain according to SEQ ID No.:
1 conjugation and labeling on affinity to 4Ig-B7H3.

| | ka | kd | KD | t ½ |
|---|---|---|---|---|
| Sample | (1/Ms) | (1/s) | (M) | (s) |
| 175Lu-DTPA-8H9-antibody (CAR1.4) | 1.99E+04 | 1.52E−04 | 7.64E−09 | 4.55E+03 |
| 175Lu-DTPA-8H9-antibody (CAR3) | 1.46E+04 | 2.37E−04 | 1.62E−08 | 2.92E+03 |
| 175Lu-DTPA-8H9-antibody (CAR3.6) | 1.44E+04 | 2.67E−04 | 1.85E−08 | 2.60E+03 |
| 175Lu-DTPA-8H9-antibody (CAR6.1) | 1.42E+04 | 4.81E−04 | 3.40E−08 | 1.44E+03 |
| 175Lu-DOTA-8H9-antibody (CAR2.6) | 1.49E+04 | 2.34E−04 | 1.57E−08 | 2.96E+03 |
| 175Lu-DOTA-8H9-antibody (CAR6.3) | 1.33E+04 | 2.60E−04 | 1.95E−08 | 2.67E+03 |
| 127I-Humanized-8H9-antibody | 1.12E+05 | 2.68E−05 | 2.40E−10 | 2.58E+04 |
| 127I-8H9-antibody P76501A | 6.61E+04 | 4.40E−05 | 6.66E−10 | 1.58E+04 |
| YMS1(hu8H9/Humanized 8H9- | 6.61E+04 | 4.49E−05 | 6.79E−10 | 1.54E+04 |
| antibody) | | | | |
| hu8H9-3.1 | 2.83E+04 | 2.51E−04 | 8.87E−09 | 2.77E+03 |
| 8H9 (8H9-antibody) P76501A | 2.86E+04 | 9.11E−05 | 3.18E−09 | 7.61E+03 |
| 8H9 (S219) | 2.79E+04 | 8.73E−05 | 3.13E−09 | 7.94E+03 |

CAR = chelator-to-antibody ratio;
DTPA = p-SCN-Bn-CHX-A″-DTPA;
DOTA = p-SCN-Bn-DOTA;
kₐ = association constant;
k_{d} = dissociation constant;
K_{D} = equilibrium dissociation constant;
T½ = half-life

The results from Tables 3 and 4 show that after the conjugation of 8H9 antibody comprising a light chain according to SEQ ID No.: 2 and heavy chain according to SEQ ID No.: 1 with p-SCN-Bn-CHX-A″-DTPA or p-SCN-Bn-DOTA the conjugated products bind to 21g- and 41g-B7H3. Lower chelator to antibody ratios (CAR) resulted in higher affinities of the conjugated 8H9-antibody comprising a light chain according to SEQ ID No.: 2 and heavy chain according to SEQ ID No.: 1 to B7H3. Conjugation to p-SCN-Bn-DOTA showed little impact on the binding to B7H3 and affinities comparable to unconjugated antibodies were obtained. It is noted that the kinetic data from the 41g-B7H3 is more reliable, since the high binding observed with unconjugated 8H9-antibody comprising a light chain according to SEQ ID No.: 2 and heavy chain according to SEQ ID No.: 1 in the 21g-B7H3 set is beyond the fitting capabilities of the Biacore T200 instrument.

In this study it is shown that DTPA and DOTA conjugated 8H9 antibodies bind to 21g- and 41g-B7H3. The degree of conjugation (conjugate-antibody ratio-CAR) and labeling, as assessed by SPR, influence the affinity of 8H9 antibody comprising a light chain according to SEQ ID No.: 2 and heavy chain according to SEQ ID No.: 1 to recombinant human B7H3 protein.

### Example 5

Immunoreactivity Results

Antigen (B7H3) conjugated streptavidin beads were produced. Specific bead production batches are described in Table 7A. Immunoreactivity assays were performed on the 177Lu-8H9 antibody comprising a light chain according to SEQ ID No.: 2 and heavy chain according to SEQ ID No.: 1 derivatives. Results are summarized in Table 7B

TABLE 7A
B7H3-bead production summaries.

| B7H3-bead production | Study 1 | | Study 2 | | Study 3 | |
|---|---|---|---|---|---|---|
| Starting mass of B7H3 | 200 μg (one | | 400 | μg | 400 | μg |
| | vial) | | | | | |
| Mass of streptavidin beads | 14 | mg | 14 | mg | 14 | mg |
| Supernatant conc. | 0.0328 | μg/mL | | | | |
| Final B7H3 concentration | 0.5 | mg/mL | 0.5 | mg/mL | 0.5 | mg/ml |
| theoretical (70% | | | | | | |
| biotinylated) | | | | | | |
| Final B7H3 concentration | 0.36 | mg/ml | — | | — | |
| estimated (49% lost#) | | | | | | |
| Final bead concentration | 50 | mg/mL | 25 | mg/mL | 50 | mg/mL |
| Final B7H3/Bead, theoretical | 10 | μg/mg | 20 | μg/mg | 20 | μg/mg |

| | | | | |
|---|---|---|---|---|
| Final B7H3/Bead, estimated | 7.26 | μg/mg | — | — |

TABLE 7B
Immunoreactivity results summary

| | DTPA- | DOTA- | DOTA- | DTPA- | DTPA- | DTPA- | DOTA- |
|---|---|---|---|---|---|---|---|
| | CAR1.4 | Invicro | Invicro | CAR1.4 | CAR3.6 | CAR6.1 | CAR6.3 |
| Total binding, % | 61.4 ± 2.3 | 86.7 ± 1.9 | 90.6 ± 3.6 | 94.6 ± 3.3 | 91.7 ± 1.6 | 89.0 ± 3.2 | 93.8 ± 1.5 |
| Non-specific | 2.1 ± 0.1 | 0.4 ± 0.0 | 1.6 ± 0.3 | 0.2 ± 0.0 | 0.3 ± 0.0 | 0.2 ± 0.1 | 1.4 ± 0.3% |
| binding, % | | | | | | | |
| Lost ACBs, % | 3.9 ± 7.4 | 4.3 ± 1.4 | 4.4 ± 2.6 | 2.4 ± 2.7 | 3.7 ± 0.5 | 5.7 ± 2.2 | 4.2 ± 1.5 |
| Immuno- | 59.3 | 86.3 | 89.0 | 94.4 | 91.4 | 88.7 | 92.5 |
| reactivity, % | | | | | | | |
| Mass of B7H3, μg | ≥7.5 μg | ≥7.5 μg | ≥7.0 μg | ≥7.0 μg | ≥7.0 μg | ≥7.0 μg | 20 μg |

### Example 6

Effect of Conjugation and Labeling on Binding Affinity

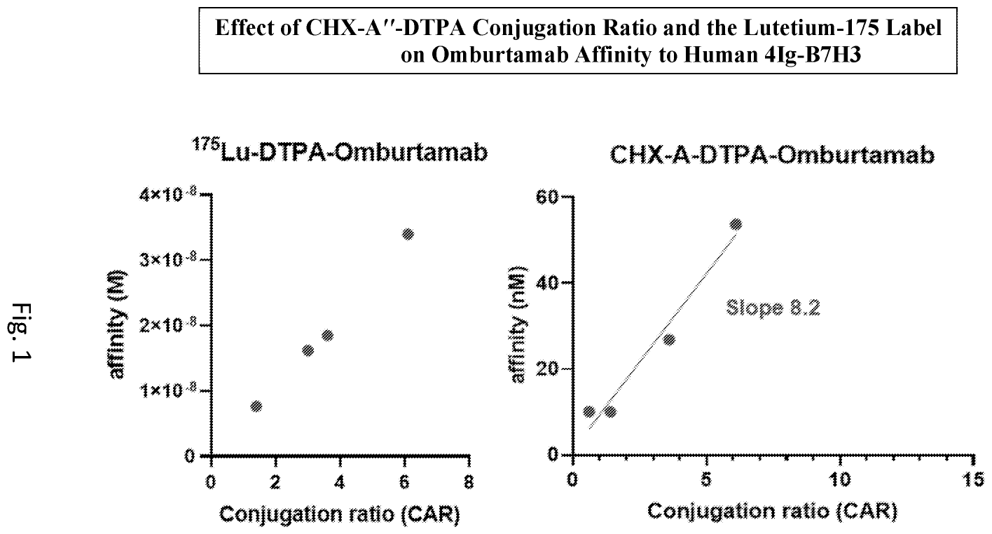

The in vitro binding affinity of 8H9 antibody comprising a light chain according to SEQ ID No.: 2 and heavy chain according to SEQ ID No.: 1 for recombinant human B7-H3 protein (21g and 41g isoforms; 41g is the dominant isoform) was compared for naked, chelated, and lutetium-175-labeled 8H9 antibody using SPR. Lower conjugation ratios of CHX-A″-DTPA resulted in higher affinity of the 8H9 antibody and 175Lu-DTPA-8H9 antibody to 41g-B7-H3 and 21g-B7-H3 (FIG. 1; Table 8). Labeling of the conjugates with cold lutetium-175 did not further change the 8H9 antibody binding affinity for B7-H3 protein (FIG. 1; Table 9), and labeling with iodine-127 did not affect binding affinity.

TABLE 8
Effect of Chelator-to-Antibody Ratio on 8H9 antibody comprising a
light chain according to SEQ ID No.: 2 and heavy chain according
to SEQ ID No.: 1 Binding Kinetics after Conjugation with CHX-A″-DTPA

| | | | | |
|---|---|---|---|---|
| | kₐ | k_{d} | K_{D} | t½ |
| Sample | (1/Ms) | (1/s) | (M) | (sec) |

human 4Ig-B7-H3 (dominant isoform)

| | | | | |
|---|---|---|---|---|
| CHX-A″-DTPA (CAR1.4) | 1.77E+04 | 1.77E−04 | 1.00E−08 | 3.91E+03 |
| CHX-A″-DTPA (CAR3.6) | 1.41E+04 | 3.79E−04 | 2.69E−08 | 1.83E+03 |
| CHX-A″-DTPA (CAR6.1) | 1.66E+04 | 8.96E−04 | 5.38E−08 | 7.74E+02 |
| conj-CHX-A″-DTPA (CAR0.6) | 2.16E+04 | 2.18E−04 | 1.01E−08 | 3.18E+03 |

human 2Ig-B7-H3

| | | | | |
|---|---|---|---|---|
| CHX-A″-DTPA (CAR1.4) | 2.52E+04 | 4.88E−05 | 1.93E−09 | 1.42E+04 |
| CHX-A″-DTPA (CAR3.6) | 1.62E+04 | 1.54E−04 | 9.46E−09 | 4.51E+03 |
| CHX-A″-DTPA (CAR 6.1) | 2.01E+04 | 4.89E−04 | 2.43E−08 | 1.42E+03 |
| conj-CHX-A″-DTPA (CAR0.6) | 2.75E+04 | 3.36E−05 | 1.22E−09 | 2.06E+04 |

CAR = chelator-to-antibody ratio; DTPA = p-SCN-Bn-CHX-A″-DTPA; kₐ = association constant; k_{d} = dissociation constant; K_{D} = equilibrium dissociation constant; t½ = half-life.

TABLE 9
Effect of Chelator-to-Antibody Ratio on 8H9 antibody
Binding Kinetics after Conjugation with CHX-A″-DTPA
and Labeling with Lutetium-175 or lodine-127

| | | | | |
|---|---|---|---|---|
| | kₐ | k_{d} | K_{D} | t½ |
| Sample | (1/M s) | (1/s) | (M) | (sec) |

human 4Ig-B7-H3 (dominant isoform)

| | | | | |
|---|---|---|---|---|
| ¹⁷⁵Lu-DTPA-8H9 | 1.99E+04 | 1.52E−04 | 7.64E−09 | 4.55E+03 |
| antibody (CAR1.4) | | | | |
| ¹⁷⁵Lu-DTPA-8H9 | 1.46E+04 | 2.37E−04 | 1.62E−08 | 2.92+03 |
| antibody (CAR3) | | | | |
| ¹⁷⁵Lu-DTPA-8H9 | 1.44E+04 | 2.67E−04 | 1.85E−08 | 2.60E+03 |
| antibody (CAR3.6) | | | | |
| ¹⁷⁵Lu-DTPA-8H9 | 1.42E+04 | 4.81E−04 | 3.40E−08 | 1.44E+03 |
| antibody (CAR6.1) | | | | |
| ¹²⁷I-8H9 | 6.61E+04 | 4.40E−05 | 6.66E−10 | 1.58E+04 |
| antibody | | | | |

human 2Ig-B7-H3

| | | | | |
|---|---|---|---|---|
| ¹⁷⁵Lu-DTPA-8H9 | 3.16E+04 | 6.87E−06 | 2.17E−10 | 1.01E+05 |
| antibody (CAR1.4) | | | | |
| ¹⁷⁵Lu-DTPA-8H9 | 1.80E+04 | 3.23E−05 | 1.80E−09 | 2.14E+04 |
| antibody (CAR3) | | | | |
| ¹⁷⁵Lu-DTPA-8H9 | 1.65E+04 | 3.86E−05 | 2.34E−09 | 1.80E+04 |
| antibody (CAR3.6) | | | | |
| ¹⁷⁵Lu-DTPA-8H9 | 1.46E+04 | 1.06E−04 | 7.21E−09 | 6.57E+03 |
| antibody (CAR6.1) | | | | |
| ¹²⁷I- 8H9 | 1.27E+05 | 6.24E−07 | 4.91E−12 | 1.11E+06 |
| antibody | | | | |

CAR = chelator-to-antibody ratio; DTPA = p-SCN-Bn-CHX-A″-DTPA; kₐ = association constant; k_{d} = dissociation constant; K_{D} = equilibrium dissociation constant; t½ = half-life.

### Example 7

In Vivo Proof of Concept for Mice Treated with a 177Lu DTPA 8H9 Antibody (CAR3) Comprising a Light Chain According to SEQ ID No.: 2 and Heavy Chain According to SEQ ID No.: 1

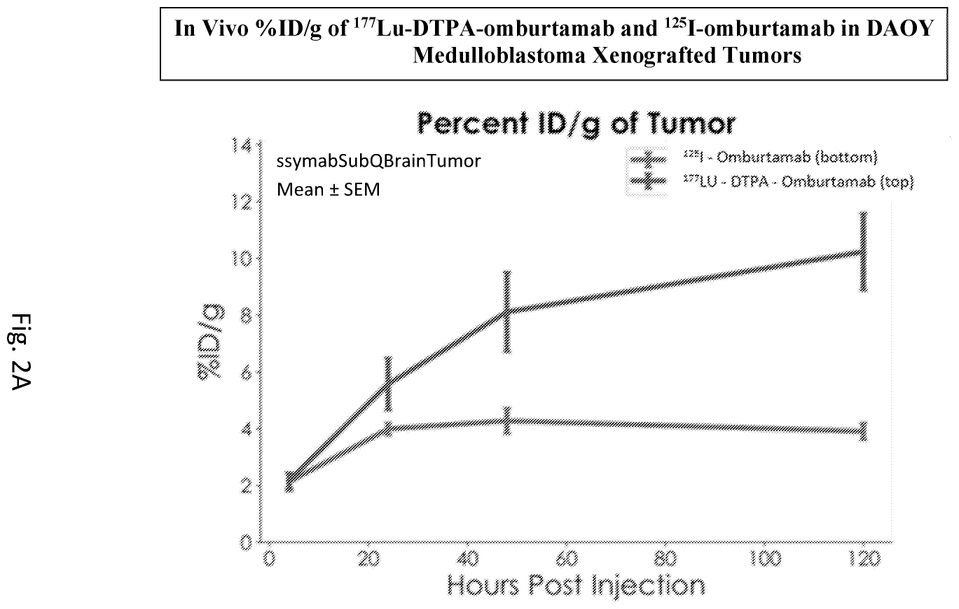

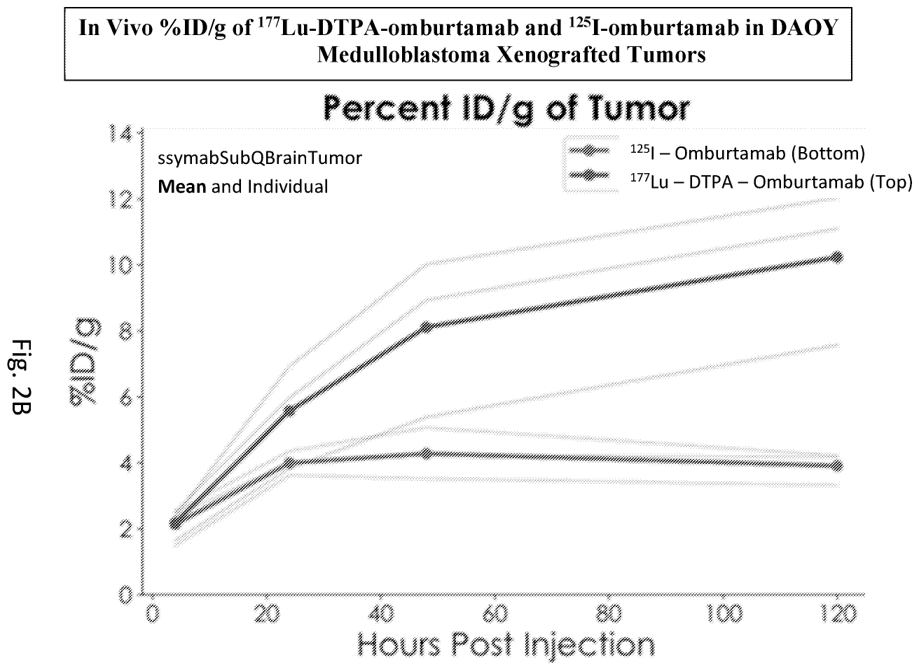

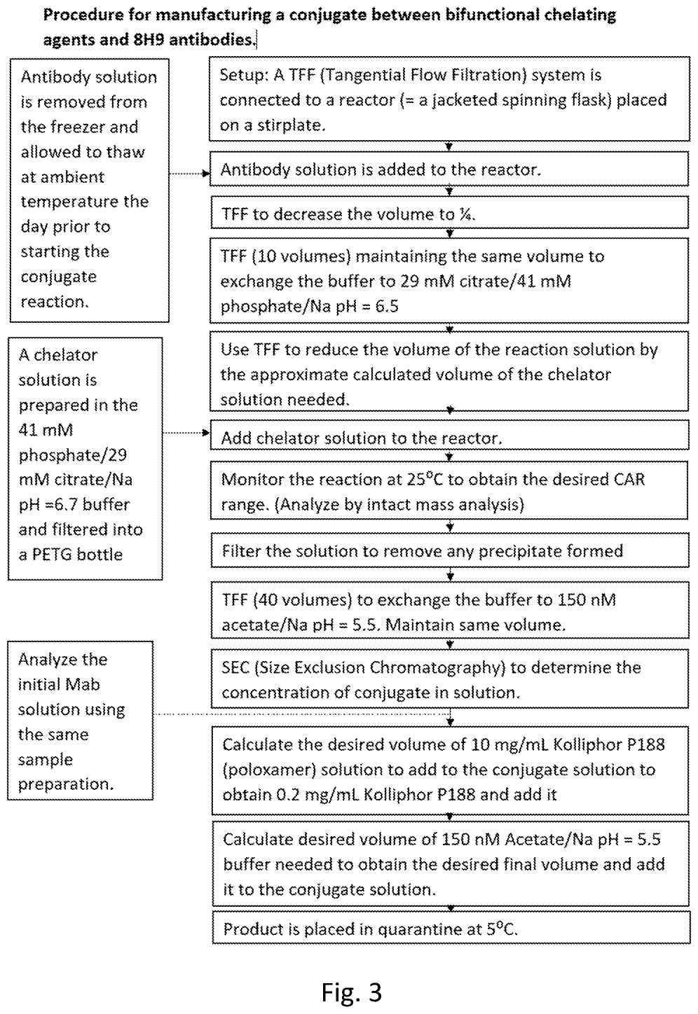

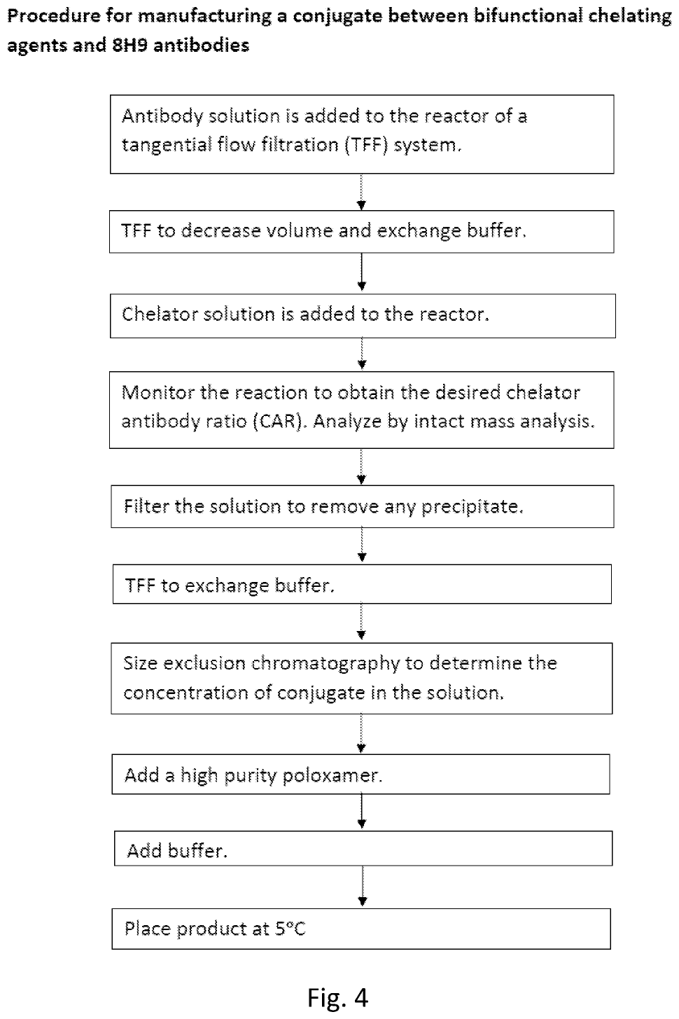

Proof-of-concept tumor targeting was demonstrated in athymic nude mice bearing B7 H3-expressing medulloblastoma xenografts. Representative results are shown in FIGS. 2A and 2B. Mice given a single intravenous (IV) dose showed accumulation of ¹⁷⁷Lu DTPA 8H9 antibody (CAR3) in tumors. The 8H9 antibody comprised a light chain according to SEQ ID No.: 2 and heavy chain according to SEQ ID No.: 1. Accumulation was compared to that of ¹²⁵I 8H9 antibody, an ¹³¹I 8H9 antibody analogue used in this study due to its suitability for imaging purposes. Over 120 hours, accumulation of ¹⁷⁷Lu DTPA-8H9 antibody in the tumor was greater than that observed with ¹²⁵I 8H9 antibody (FIGS. 2A and 2B).

### Example 8

Image Analysis and Dosimetry for Rats Treated with ¹⁷⁷Lu-DTPA-8H9 or 177Lu DOTA-Omburtamab (CAR 6.3) Antibody Comprising a Light Chain According to SEQ ID No.: 2 and Heavy Chain According to SEQ ID No.: 1

Radiation dosimetry estimated were determined from rats treated IT with a high dose of 500 μCi/animal ¹⁷⁷Lu-DTPA-8H9 or ¹⁷⁷Lu-DOTA-omburtamab (CAR 6.3) antibody comprising a light chain according to SEQ ID No.: 2 and heavy chain according to SEQ ID No.: 1 (CAR 3).

Reconstructed SPECT images were generated in units of activity. Namely, the values assigned to the voxels (volume elements) comprising the 3D reconstructed SPECT images were in units of μCi or equivalent. Reconstructed images were co-registered to one another, resampled to 0.3 mm3 voxels, and cropped to a uniform size prior to analysis.

The brain ROI (regions of interest) was generated with aid of the 3D Brain Atlas tool. After initial placement of the atlas, the ROI was manually edited to match its appearance on CT. The heart, liver, lungs and spleen were defined by manually fitting ellipsoids of fixed volume to the respective organs in each image. The kidney ROIs (right and left combined) were defined by ellipsoids of fixed volume determined from the CT image.

The spinal cord (SC) was defined using connected thresholding on CT and then split into four regions based on identification of vertebrae: cervical SC, upper thoracic SC, lower thoracic SC and lumbar SC. The humerus was defined using connected thresholding on CT with the proximal epiphysis segmented as trabecular and the remaining humerus segmented as cortical. Deep and superficial cervical lymph nodes were defined by two fixed volume spherical ROIs placed over the left and right regions on each image.

A liver specific calibration factor was derived from the whole organ activity measured in co-acquired SPECT and planar scans. This factor was used to convert planar values to activity units while accounting for attenuation correction. The whole organ liver volume was measured from an individual SPECT/CT scan for the purposes of % ID/g calculations. Results were in units of percent injected dose and percent injected dose per gram.

Maximum intensity projections (MIP) images were generated for each animal at 4 scheduled time points being 1, 24, 144 and 264 h respectively. Images were converted to units of injected dose per gram tissue (% ID/g) and scaled from 0 to 7,5% ID/g.

For each region of interest, plots of the mean activity over time, per region, were generated for each rat treated with the high dose of 177Lu-DTPA-8H9 antibody. The area under the curve (AUC) was calculated to arrive at the mean residence time (MRT). The MRT is defined as the average residence time of the labeled test article in the tissue of interest. The AUC was generated using trapezoidal integration of the four data points through the origin (area under the time activity curve).

The contribution to the mean residence time following the last imaging time point (hour 264) was estimated by fitting the data to a single or a biexponential model. When both the single and biexponential models assumed greater activity than by physical decay, a physical decay only model was used in place. Physical decay only assumes no further biological clearance or accumulation occurred and radioactive decay is extrapolated out to infinity. For the brain, % ID human was considered equivalent to % ID rat and MRT value was calculated as described above. For all other source organs, human MRT values were computed by multiplying the rat MRT values by the human organ weight to bodyweight ratio and dividing by the rat organ weight (determined from the ROI, assuming a density of 1 g/mL) to bodyweight ratio. Intrathecal ¹⁷⁷Lu DTPA-8H9 antibody (- CAR 3) estimated mean residence times (MRT) for adults and children are included in Table 10. MRT are greatest in the liver, cortical bone, and brain with respective MRT of 16.61 h, 7.08 h, and 4.43 h in the adult male and similar MRT in adult female and pediatric subjects. MRT in the liver are longer in children than adults with estimates of 20.21 h in 5-year old children (both sexes) and 22.23 h in 1-year old children (both sexes).

The three organs receiving the greatest radiation absorbed dose are summarized in Table 11A and 11B. For all subject estimates, the liver received the greatest absorbed dose, varying from 0.83 mGy/MBq in the adult male to 5.90 mGy/MBq in one-year old children (both sexes). Osteogenic cells received the second greatest dose (0.54 mGy/MBq in adult females to 4.05 mGy/MBq in 1-year old males) followed by the kidneys (0.32 mGy/MBq in adult males to 1.79 mGy/MBq in both sexes of 1-year old subjects). Adult females received greater absorbed dose in the liver and kidneys compared to adult males, while adult males received slightly greater dose to osteogenic cells than females. Radiation absorbed doses were nearly identical between sexes for liver, osteogenic cells, and kidneys for pediatric subjects. Total body effective doses are also presented in Table 12A and 12B. The estimated total body effective dose is 0.13 mSv/MBq in adult males, 0.18 mSv/MBq in adult females, 0.50 mSv/MBq in 5-year old subjects, and 0.97 -0.98 mSv/MBq in 1-year old subjects.

TABLE 10
Mean Residence Times for ¹⁷⁷Lu-DTPA-8H9 antibody (CAR 3)

| | Adult | | 5 y.o. | | 1 y.o. | |
|---|---|---|---|---|---|---|
| Source Organ | Male (h) | Female (h) | Male (h) | Female (h) | Male (h) | Female (h) |
| Brain | 4.43 | 4.43 | 4.43 | 4.43 | 4.43 | 4.43 |
| Heart | 1.83 | 1.83 | 1.83 | 1.83 | 1.83 | 1.83 |
| Kidneys | 1.08 | 1.17 | 1.48 | 1.48 | 1.79 | 1.79 |
| Liver | 16.61 | 15.72 | 20.21 | 20.21 | 22.23 | 22.23 |
| Cortical bone | 7.08 | 6.27 | 6.25 | 6.25 | 5.52 | 5.52 |
| Trabecular | 2.89 | 2.55 | 2.52 | 2.52 | 2.30 | 2.30 |
| bone | | | | | | |
| Total body | 122.50 | 122.50 | 122.50 | 122.50 | 122.50 | 122.50 |
| Remainder | 88.57 | 90.52 | 85.78 | 85.78 | 84.40 | 84.40 |

TABLE 11A
Summary of the Organs Receiving the
Highest Absorbed Doses (mGy/mCi)

| | ¹⁷⁷Lu-DTPA-8H9 antibody | | | | | |
|---|---|---|---|---|---|---|
| | Absorbed dose, mGy/mCi (mean) | | | | | |
| | Adult | | Pediatric, 5 y.o. | | Pediatric, 1 y.o. | |
| | Male | Female | Male | Female | Male | Female |
| Liver | 30.62 | 37.15 | 115.50 | 115.50 | 218.30 | 218.30 |
| Osteogenic | 21.90 | 19.94 | 70.36 | 69.38 | 149.85 | 147.38 |
| cells | | | | | | |
| Kidneys | 11.80 | 14.31 | 44.40 | 44.40 | 84.18 | 83.99 |

TABLE 11B
Summary of the Organs Receiving the
Highest Absorbed Doses (mGy/MBq)

| | ¹⁷⁷Lu-DTPA-8H9 antibody | | | | | |
|---|---|---|---|---|---|---|
| | Absorbed dose, mGy/MBq (mean) | | | | | |
| | Adult | | Pediatric, 5 y.o. | | Pediatric, 1 | |
| | Male | Female | Male | Female | Male | Female |
| Liver | 0.83 | 1.00 | 3.12 | 3.12 | 5.90 | 5.90 |
| Osteogenic | 0.59 | 0.54 | 1.90 | 1.88 | 4.05 | 3.98 |
| cells | | | | | | |
| Kidneys | 0.32 | 0.39 | 1.20 | 1.20 | 2.28 | 2.27 |

Total body effective dose (mSv/MBq)

| | | | | | |
|---|---|---|---|---|---|
| 0.13 | 0.18 | 0.50 | 0.50 | 0.98 | 0.97 |

Table 12 Shows the Complete ¹⁷⁷Lu-DTPA-8H9 Antibody (CAR 3) Dosimetry Estimates for an Adult Male (73 kg) and Table 13 Shows the Complete ¹⁷⁷Lu-DOTA-8H9 Antibody (CAR 6.3) Dosimetry Estimates for an Adult Male (73 kg)

TABLE 12
¹⁷⁷Lu-DTPA-8H9 antibody (CAR3) dosimetry results for an
adult male (73 kg). Estimates derived from imaging of
Sprague Dawley rats and scaled using the % kg/g method.

| | 177Lu-DTPA- | 177Lu-DTPA- | cGy per 25 mCi of |
|---|---|---|---|
| | Omburtamab | Omburtamab | 177Lu-DTPA- |
| | (CAR 3) (mean), | (CAR 3) (mean), | Omburtamab |
| Target Organ | mGy/mCi | mGy/MBq | (CAR 3) |
| Adrenals | 5.01 | 0.14 | 12.53 |
| Brain | 10.18 | 0.28 | 25.45 |
| Breasts | — | — | — |
| Esophagus | 4.50 | 0.12 | 11.24 |
| Eyes | 4.23 | 0.11 | 10.57 |
| Gallbladder Wall | 5.19 | 0.14 | 12.98 |
| Heart Wall | 10.34 | 0.28 | 25.85 |
| Kidneys | 11.80 | 0.32 | 29.51 |
| Left colon | 4.44 | 0.12 | 11.10 |
| Liver | 30.62# | 0.83# | 76.54# |
| Lungs | 4.43 | 0.12 | 11.07 |
| Osteogenic Cells | 21.90 | 0.59 | 54.76 |
| Ovaries | — | — | — |
| Pancreas | 4.63 | 0.13 | 11.56 |
| Prostate | 4.35 | 0.12 | 10.88 |
| Rectum | 4.37 | 0.12 | 10.92 |
| Red Marrow | 4.95 | 0.13 | 12.38 |
| Right colon | 4.55 | 0.12 | 11.38 |
| Salivary Glands | 4.32 | 0.12 | 10.79 |
| Small Intestine | 4.45 | 0.12 | 11.12 |
| Spleen | 4.38 | 0.12 | 10.95 |
| Stomach Wall | 4.49 | 0.12 | 11.22 |
| Testes | 4.19 | 0.11 | 10.48 |
| Thymus | 4.38 | 0.12 | 10.95 |
| Thyroid | 4.30 | 0.12 | 10.75 |
| Urinary Bladder Wall | 4.34 | 0.12 | 10.85 |
| Uterus | — | — | — |
| Total Body | 5.74 | 0.16 | 14.34 |
| Total Body Effective | | 0.13 | |
| Dose | | (mSv/MBq) | |

#Dose limiting organ

TABLE 13
¹⁷⁷Lu-DOTA-8H9 antibody (CAR 6.3) dosimetry estimates for
an adult male (73 kg). Estimates derived from imaging of
Sprague Dawley rats and scaled using the % kg/g method.

| | 177Lu-DOTA- | 177Lu-DOTA- | cGy per 25 mCi of |
|---|---|---|---|
| | Omburtamab | Omburtamab | 177Lu-DOTA- |
| | (CAR 6.3) (mean), | (CAR 6.3) (mean), | Omburtamab |
| Target Organ | mGy/mCi | mGy/MBq | (CAR 6.3) |
| Adrenals | 4.57 | 0.12 | 11.42 |
| Brain | 11.43 | 0.31 | 28.57 |
| Breasts | — | — | — |
| Esophagus | 4.06 | 0.11 | 10.15 |
| Eyes | 3.83 | 0.10 | 9.59 |
| Gallbladder Wall | 4.76 | 0.13 | 11.90 |
| Heart Wall | 8.34 | 0.23 | 20.86 |
| Kidneys | 9.63 | 0.26 | 24.08 |
| Left colon | 4.01 | 0.11 | 10.03 |
| Liver | 30.62# | 0.83# | 76.56# |
| Lungs | 4.02 | 0.11 | 10.04 |
| Osteogenic Cells | 15.51 | 0.42 | 38.77 |
| Ovaries | — | — | — |
| Pancreas | 4.19 | 0.11 | 10.48 |
| Prostate | 3.93 | 0.11 | 9.82 |
| Rectum | 3.94 | 0.11 | 9.86 |
| Red Marrow | 4.04 | 0.11 | 10.10 |
| Right colon | 4.12 | 0.11 | 10.30 |
| Salivary Glands | 3.91 | 0.11 | 9.77 |
| Small Intestine | 4.02 | 0.11 | 10.05 |
| Spleen | 3.95 | 0.11 | 9.88 |
| Stomach Wall | 4.06 | 0.11 | 10.14 |
| Testes | 3.78 | 0.10 | 9.46 |
| Thymus | 3.95 | 0.11 | 9.87 |
| Thyroid | 3.88 | 0.10 | 9.70 |
| Urinary Bladder Wall | 3.92 | 0.11 | 9.79 |
| Uterus | — | — | — |
| Total Body | 5.17 | 0.14 | 12.93 |
| Total Body Effective | | 0.12 | |
| Dose | | (mSv/MBq) | |

#Dose limiting organ

### Example 9

Procedure for Manufacturing a Conjugate Between P-Scn-Bn-Chx-A″-Dtpa, and an 8H9 Antibody Comprising a Light Chain According to Seq Id No.: 2 and Heavy Chain According to Seq Id No.: 1.

p-SCN-Bn-CHX-A″-DTPA is a bifunctional chelating agent that can be conjugated to lysine side chains in a random lysine conjugation process. The final conjugate can be labeled with the beta emitter, Lu-177, for radioimmunotherapy.

Tangential flow filtration (TFF) is used to reduce the volume of the antibody solution to one fourth. TFF (10 volumes) is used to exchange the buffer to 41 mM phosphate/29 mM citrate/Na pH=6.5. A solution of p-SCN-Bn-CHX-A″-DTPA in the same buffer is added straight. The reaction is kept at 25° C. while being monitored for CAR value. Once the target CAR value is achieved, the reaction is filtered to remove any precipitate that has formed. TFF (40 volumes) is used to exchange the buffer to 15 mM acetate/Na pH=5.5. The volume and concentration of conjugate is determined. Solutions of Poloxamer 188 and final buffer are added to achieve the target concentrations of Poloxamer 188 and conjugate.

1) Equipment, Raw Material and mAb Preparation:
The main reactor is a jacketed spinner flask. Reactor size should be chosen on the basis of the total volume of the reaction to be placed into the reactor. The day prior to starting the conjugation reaction the Mab solution is removed from the freezer, and the solution is allowed to thaw at ambient temperature.
Obtain the gross weight of the bottle, cap and solution. The solution may be placed at 5° C. until it is needed.

2) Solution Preparations:
Prepare the following solutions:
   0.1 N NaOH Cleaning solution
   1.0 M NaOH solution
   Calibrate a combination pH electrode at pH=4 and 7
   29 mM Citrate/41 mM Phosphate/Na pH=6.5 buffer
   150 mM Acetate/Na pH=5.5 buffer

3) TFF Cassette Cleaning:
In a chemical fume hood, prepare a hotplate/stirrer. Pour 0.1 N NaOH Cleaning solution into a flask and add a stirbar. Heat the solution to 45° C. Maintain a temperature of 40-50° C. during the cleaning step. Replenish the cleaning solution as needed.
Connect transfer tubing to feed, permeate, and retentate ports of a TFF cassette. The feed lines run through a peristaltic pump before being placed in the above flask. The permeate and retentate lines shall be placed into their own waste containers. Clean the cassette by pumping at least 100 mL solution through the permeate line. When complete, seal the cassette with cleaning solution inside.
Drain the liquid from the tubing. Use a syringe to blow out residual liquid. Connect the tubing to itself using male-to-male fittings. Only residual cleaning solution will remain inside.

4) Reactor and TFF Cassette Setup:
Clean a biological safety cabinet with 70% isopropyl alcohol. Setup the reactor on a stirplate. Turn on the stirplate to verify the stirrer is aligned. Turn the stirplate back off until needed.
Connect the reactor to the circulating water bath. Turn the bath on and set to 25° C. Verify water circulates around the jacketed reactor. The reactor is now ready for use. Setup a TFF cassette with transfer tubing and peristaltic pump. Put the feed line into water. Put the permeate and retentate lines into waste. Run water through in 100 mL increments. Test the permeate effluent by pH paper. Once the pH=6-7, run another 100 mL more water through. Drain the liquid from the tubing. Use a syringe to blow out residual liquid. The tubing and cassette are now ready for use.

5) Reaction:
Connect the TFF system to the reactor. Add 8H9 antibody comprising a light chain according to SEQ ID No.: 2 and heavy chain according to SEQ ID No.: 1 to the reactor. The addition may be performed in increments in conjunction with the steps as TFF is performed to decrease the volume. Based on a solution density of 1.0 g/mL, the mass in grams equals the volume in mL.
Perform TFF to decrease the initial volume to ˜¼.
Use TFF to perform ten volume exchanges using 29 mM Citrate/41 mM
Phosphate/Na pH=6.5 buffer. Maintain the volume at approximately the same level. Calibrate a combination pH electrode at pH=4 and 7.
In a PETG bottle, prepare a 20 mg/mL p-SCN-Bn-CHX-A″-DTPA Solution by dissolving p-SCN-Bn-CHX-A″-DTPA in 29 mM Citrate/41 mM Phosphate/Na pH=6.5 buffer. Mix the solution. Measure the pH. Add 1.0 M NaOH solution in small increments to increase the pH to 6.45-6.55. Record the final pH. Using a syringe, filter the solution through a 0.22 μm PVDF filter into a PETG bottle. Rinse the original container with 29 mM Citrate/41 mM Phosphate/Na pH=6.5 buffer. Filter the rinse through the same filter into the PETG bottle.
Calculate the volume of 20 mg/mL p-SCN-Bn-CHX-A″-DTPA solution needed to add to the reactor.
Use TFF to reduce the volume of the reaction solution by the approximate volume of p-SCN-Bn-CHX-A″-DTPA solution calculated above.
Add the 20 mg/mL p-SCN-Bn-CHX-A″-DTPA solution calculated directly to the reactor.

6) Monitoring:
Monitor the reaction as needed to obtain a CAR value in the desired range. To analyze, add 5 μL of reaction solution to 45 μL 10 M NH₃/NH₄Cl buffer. Incubate 30 min at 37° C. Add 50 μL 1% formic acid in water. Analyze by intact mass analysis.

7) RXN Work-Up:
Filter the solution through a 10 μm polypropylene filter into a PETG bottle or labtainer.
Rinse the reactor with 29 mM Citrate/41 mM Phosphate/Na pH=6.5 buffer. Filter this solution through the above 10 μm filter and into the same container.
Filter the solution through a 0.22 μm PVDF filter into a clean reactor.
Rinse the container with 29 mM Citrate/41 mM Phosphate/Na pH=6.5 buffer. Filter this solution through the above 0.22 μm PVDF filter and into the reactor.
Rinse the TFF cassette(s) by pumping Water (Milli-Q) through the permeate for each cassette.
Use TFF to perform forty volume exchanges using 150 mM acetate/Na pH=5.5 buffer. Maintain the volume at approximately the same level throughout.
When TFF is complete, transfer the reaction solution into a tared PETG bottle or labtainer. Use a syringe to blow residual liquid in the lines into the reactor.

8) Final Formulation:
Using SEC chromatography, determine the concentration of conjugate in solution. To analyze, add 10 μL of solution to 90 μL of water. Analyze the initial Mab solution using the same sample preparation. An equation is used to determine the amount of solution to calculate the concentration of conjugate in solution.
The desired final volume based on a target concentration of 2.0 mg/mL can be calculated.
Prepare a 10.0 mg/mL Kolliphor P188 (high purity poloxamer) solution by dissolving Kolliphor P188 BIO in 150 mM Acetate/Na pH=5.5 buffer in a PETG bottle. Filter the solution through a 0.22 μm PVDF filter into a PETG bottle.
Use an equation to calculate the desired volume of 10 mg/mL Kolliphor P188 solution to add to the conjugate solution to obtain 0.2 mg/mL Kolliphor P188.
Add the volume into the conjugate solution (Vol_{conjugate}).
Calculate the desired volume of 150 mM Acetate/Na pH=5.5 buffer needed to obtain the desired final volume, and add it to the conjugated solution.
Product is placed in quarantine at 5° C.A final yield of 84% was obtained at a scale of 1.95 g.

Discussion and Conclusions

DTPA and DOTA conjugated 8H9 antibodies, including ¹⁷⁷Lu-DTPA-8H9 antibody, are being developed for the treatment of B7-H3-positive tumors. Results from a substantial amount of in vitro work demonstrated expression of B7-H3 on a broad spectrum of cancer cell types, including medulloblastoma, and selective binding of 8H9 antibody to B7-H3, including the membrane-bound protein. The minimal binding in normal tissues demonstrated 8H9 antibody's potential as an effective mechanism for delivering a radioactive payload to tumors while minimizing impact to normal tissues. Of particular note, B7-H3 immunostaining with 8H9 antibody was negative in normal tissues, including brain and bone marrow, in both cynomolgus monkeys (the species used in safety assessments) and humans.

Binding kinetics as measured by SPR showed that the conjugation of the DOTA or DTPA linker and optionally lutetium-177 radiolabel resulted in conjugated 8H9 antibodies capable of binding to the target antigen (ie, 41g-B7-H3). A CAR of approximately 3 was identified as appropriate for delivering the necessary level of radioactivity without negatively impacting the binding affinity.

¹⁷⁷Lu-DTPA-8H9 antibody was shown to target and accumulate in B7-H3 expressing medulloblastoma tumor tissue as measured by SPECT/CT (Single Photon Emission Computed Tomography/Computed Tomography) imaging. ¹⁷⁷Lu-DTPA-8H9 antibody has a t1/2 similar to ¹³¹I-8H9 antibody (Dash 2015), a shorter tissue irradiation range (Dash 2015; Advanced Accelerator Applications, S.r.l., 2018), and greater accumulation in tumor and tumor-to-background ratios. Therefore, the antitumor properties for ¹⁷⁷Lu-DTPA-8H9 antibody are expected to be favorable compared to ¹³¹I-8H9 antibody, a compound with demonstrated antitumor effects in humans.

Human dosimetry estimations based on biodistribution studies in rats show that ¹⁷⁷Lu-DTPA-omburtamab or ¹⁷⁷Lu-DOTA-omburtamab result in favorable normal organ exposure compared to ¹³¹I-omburtamab, which is in clinical development with no dose limiting toxicities.

In summary, the nonclinical pharmacology data supports development of DTPA and DOTA conjugated 8H9 antibodies, including ¹⁷⁷Lu-DTPA-8H9 antibody, for the treatment B7-H3-expressing tumors. Data showed the antibody selectively binds to B7-H3-expressing cancer cells. Antitumor activity of the ¹⁷⁷Lu-DTPA-8H9 antibody is suggested based on in vivo binding to DAOY medulloblastoma xenografts and substantial evidence from nonclinical and clinical experience with ¹³¹I-8H9 antibody. Taken together, the in vitro and in vivo characterization of ¹⁷⁷Lu-DTPA-8H9 antibody pharmacology, which demonstrates its potential effectiveness as a targeted radioimmunotherapy, supports the development of DTPA and DOTA conjugated 8H9 antibodies for treating B7H3 positive tumors and cancers.

The content of the ASCII text file of the sequence listing named “Substitute-Sequence-Listing-12397-2101”, having a size of 14.3 kb and a creation date of 4 Apr. 2023, and electronically submitted via EFS-Web on 7 Apr. 2023, is incorporated herein by reference in its entirety.

### REFERENCES

Ahmed M, Cheng M, Zhao Q, Goldgur Y, Cheal S M, Guo H, Larson S M, Cheung N V; “Humanized Affinity-matured Monoclonal Antibody 8H9 Has Potent Antitumor Activity and Binds to FG Loop of Tumor Antigen B7-H3”; J Biol Chem. 2015 Dec. 11; 290(50): 30018-30029.
Bailey K, Pandit-Taskar N, Humm J L, ZanZonico P, Gilheeney S, Cheung N V, Kramer K. “Targeted radioimmunotherapy for embryonal tumor with multilayered rosettes”. J Neurooncol 2019 May; 143(1): 101-106.
Blakkisrud J et al; “Biodistribution and Dosimetry Results from a Phase 1 Trial of Therapy with the Antibody-Radionucleotide Conjugate 177Lu-Lilotomab Satetraxetan” J Nucl Med 2018; 59:704-710. DOI: 10.2967/jnumed.117.195347 Dash A, Pillai M R, Knapp F F. “Production of (177)Lu for targeted radionuclide therapy: Available options”. Nucl Med Mol Imaging. 2015; 49(2):85-107.
GlaxoSmithKline. Bexxar [package insert]. U.S. Food and Drug Administration website. www.accessdata.fda.gov/drugsatfda_docs/label/2012/125011s0126lbl.pdf. 2012. Accessed December 4,2019.
Hall W C, Price-Schiavi S H, Wicks J, Rojko J L. “Tissue cross-reactivity studies for monoclonal antibodies: Predictive value and use for selection of relevant animal species for toxicity testing”. In: Cavagnaro J A, ed. Preclinical safety evaluation of biopharmaceuticals: A science-based approach to facilitating clinical trials. Hoboken, NJ: John Wiley & Sons, Inc.; 2008.
Hofman M S et al “177Lu-PSMA-617 radionuclide treatment in patients with metastatic castration-resistant prostate cancer (LuPSMA trial): a single-centre, single-arm, phase 2 study” Lancet Oncol may 2018, dx.doi.org/10.1016/51470-2045(18)30198-0.
Kramer K, Kushner B et al; A Curative Approach to Central Nervous System Metastases of Neuroblastoma; FP096 510P19-1645; 2019.
Kramer K, Pandit-Taskar N et al; A phase II study of radioimmunotherapy with intraventricular ¹³⁸I-3F8 for medulloblastoma; Pediatric Blood & CancerVolume 65, Issue 1, 2017.
Kramer et al, abstract; Safety and efficacy of intraventricular 131I-labeled monoclonal antibody 8H9 targeting the surface glycoprotein B7-H3; Neuro-Oncology; 2017.
Kramer K, Pandit-Taskar N et al; Safety and Efficacy of intraventricular 131I-Labeled Monoclonal Antibody 8H9 Targeting the Surface Glycoprotein B7-H3; V557 510P19-1597; 2019.
Leach M W, Halpern W G, Johnson C W, et al. Use of tissue cross-reactivity studies in the development of antibody-based biopharmaceuticals: history, experience, methodology, and future directions. Toxicol Pathol. 2010; 38(7):1138-66.
Merino M E, Navid F, Christensen B L, et al. Immunomagnetic purging of Ewing's sarcoma from blood and bone marrow: quantitation by real-time polymerase chain reaction. J Clin Oncol, 2001; 19:3649-3659.
Modak S, Gerald W, Cheung N K. Disialoganglioside GD2 and a novel tumor antigen: potential targets for immunotherapy of desmoplastic small round cell tumor. Med Pediatr Oncol. 2002; 39:547-551.
Modak S, Guo H F, Humm J L, Smith-Jones P M, Larson S M, Cheung N K. Radioimmunotargeting of human rhabdomyosarcoma using monoclonal antibody 8H9. Cancer Biother Radiopharm, 2005; 20:534-546.
Modak S, Kramer K, Gultekin S H, Guo H F, Cheung N K. Monoclonal antibody 8H9 targets a novel cell surface antigen expressed by a wide spectrum of human solid tumors. Cancer Res. 2001; 61:4048-4054.
Modak, S. et al “Whole Abdominopelvic Radiotherapy and Radioimmunotherapy After Complete Resection of Desmoplastic Small Round Cell Tumor (DSRCT): Major Impact on Survival.” 2019 CTOS Annual Meeting November 13-16 Tokyo, Japan Paper #22 321509. Pandit-Taskar N et al; “Biodistribution and Dosimetry of Intraventricularly Administered ¹²⁴I-Omburtamab in Patients with Metastatic Leptomeningeal Tumors” Journal of Nuclear Medicine, August, 2019 doi:10.2967/jnumed.118219576
Spectrum Pharmaceuticals, Inc. Zevalin [package insert]. U.S. Food and Drug Administration website. www.accessdata.fda.gov/drugsatfda_docs/label/2009/125019s0156.pdf. 2009. Accessed Dec. 4, 2019.
Vallabhajosula S et al; “Radioimmunotherapy of Prostate Cancer Using 90Y- and 177Lu-Labeled J591 Monoclonal Antibodies: Effect of Multiple Treatments on Myelotoxicity” Clin Cancer Res 2005;11 (19 Suppl) Oct. 1, 2005.
Xu H, Cheung I Y, Guo H F, Cheung N K. MicroRNA miR-29 modulates expression of immunoinhibitory molecule B7-H3: potential implications for immune based therapy of human solid tumors, Cancer Res, 2009; 69:6275-81.
Zhou Z, Luther N, Ibrahim G M, et al. B7-H3, a potential therapeutic target, is expressed in diffuse intrinsic pontine glioma. J Neurooncol. 2013; 111:257-264.

Sequences:
SEQ ID NO: 1:
Murine 8H9 Heavy chain
QVQLQQSGAELVKPGASVKLSCKASGYTFTNYDINWVRQR
PEQGLEWIGWIFPGDGSTQYNEKFKGKATLTTDTSSSTAY
MQLSRLTSEDSAVYFCARQTTATWFAYWGQGTLVTVSAAK
TTPPSVYPLAPGSAAQTNSMVTLGCLVKGYFPEPVTVTWN
SGSLSSGVHTFPAVLQSDLYTLSSSVTVPSSTWPSETVTC
NVAHPASSTKVDKKIVPRDCGCKPCICTVPEVSSVFIFPP
KPKDVLTITLTPKVTCVVVDISKDDPEVQFSWFVDDVEVH
TAQTQPREEQFNSTFRSVSELPIMHQDWLNGKEFKCRVNS
AAFPAPIEKTISKTKGRPKAPQVYTIPPPKEQMAKDKVSL
TCMITDFFPEDITVEWQWNGQPAENYKNTQPIMDTDGSYF
VYSKLNVQKSNWEAGNTFTCSVLHEGLHNHHTEKSLSHSP
GK
SEQ ID NO: 2:
Murine 8H9 Light Chain
DIVMTQSPATLSVTPGDRVSLSCRASQSISDYLHWYQQKS
HESPRLLIKYASQSISGIPSRFSGSGSGSDFTLVKWKIDG
SERQNGVLNSWTDQDSKDSTYSMSSTLTLTKDEYERHNSY
TCEATHKTSTSPIVKSFNRNEC
SEQ ID NO: 3:
8H9 Heavy Chain CDR-1
NYDIN
SEQ ID NO: 4:
8H9 Heavy Chain CDR-2
WIFPGDGSTQY
SEQ ID NO: 5:
8H9 Heavy Chain CDR-3
QTTATWFAY
SEQ ID NO: 6:
8H9 Light Chain CDR-1
RASQSISDYLH
SEQ ID NO: 7:
8H9 Light Chain CDR-2
YASQSIS
SEQ ID NO: 8:
8H9 Light Chain CDR-3
QNGHSFPLT
SEQ ID NO: 9:
41g-B7H3
MLRRRGSPGMGVHVGAALGALWFCLTGALEVQVPEDPVVA
LVGTDATLCCSFSPEPGFSLAQLNLIWQLTDTKQLVHSFA
EGQDQGSAYANRTALFPDLLAQGNASLRLQRVRVADEGSF
TCFVSIRDFGSAAVSLQVAAPYSKPSMTLEPNKDLRPGDT
VTITCSSYQGYPEAEVFWQDGQGVPLTGNVTTSQMANEQG
LFDVHSILRVVLGANGTYSCLVRNPVLQQDAHSSVTITPQ
RSPTGAVEVQVPEDPVVALVGTDATLRCSFSPEPGFSLAQ
LNLIWQLTDTKQLVHSFTEGRDQGSAYANRTALFPDLLAQ
GNASLRLQRVRVADEGSFTCFVSIRDFGSAAVSLQVAAPY
SKPSMTLEPNKDLRPGDTVTITCSSYRGYPEAEVFWQDGQ
GVPLTGNVTTSQMANEQGLFDVHSVLRVVLGANGTYSCLV
RNPVLQQDAHGSVTITGQPMTFPPEALWVTVGLSVCLIAL
LVALAFVCWRKIKQSCEEENAGAEDQDGEGEGSKTALQPL
KHSDSKEDDGQEIA
SEQ ID NO: 10:
2lg-B7H3
MLRRRGSPGMGVHVGAALGALWFCLTGALEVQVPEDPVVA
LVGTDATLCCSFSPEPGFSLAQLNLIWQLTDTKQLVHSFA
EGQDQGSAYANRTALFPDLLAQGNASLRLQRVRVADEGSF
TCFVSIRDFGSAAVSLQVAAPYSKPSMTLEPNKDLRPGDT
VTITCSSYRGYPEAEVFWQDGQGVPLTGNVTTSQMANEQG
LFDVHSVLRVVLGANGTYSCLVRNPVLQQDAHGSVTITGQ
PMTFPPEALWVTVGLSVCLIALLVALAFVCWRKIKQSCEE
ENAGAEDQDGEGEGSKTALQPLKHSDSKEDDGQEIA
SEQ ID NO: 11: B7H3 Epitope
IRFD
SEQ ID NO: 12:
Alternative 8H9 Heavy Chain CDR-2
WIFPGDGSTQYNEKFKG

1.-65. (canceled)

66. Antibodies or antigen binding fragments thereof conjugated to one or more chelators, wherein the chelator-to-antibody ratio (CAR) is larger than one, and wherein said antibodies or fragments are capable of binding an antigen, wherein said antigen is B7H3.

67. The antibodies or antigen binding fragments according to claim 0, wherein the chelator-to-antibody ratio (CAR) is 1.1-10.

68. The antibodies or antigen binding fragments thereof according to claim 66, wherein said antibodies or antigen binding fragments comprise at least one sequence selected from the group consisting of a heavy chain variable region CDR1 according to SEQ ID No.: 3, a heavy chain variable region CDR2 according to SEQ ID No.: 4, a heavy chain variable region CDR3 according to SEQ ID No.: 5 a light chain variable region CDR1 according to SEQ ID No.: 6, a light chain variable region CDR2 according to SEQ ID No.: 7 and a light chain variable region CDR3 according to SEQ ID No.: 8.

69. The antibodies or antigen binding fragments thereof according to claim 66, wherein said antibodies or antigen binding fragments comprise a heavy chain sequence according to SEQ ID No.: 1 and/or a light chain sequence according to SEQ ID No.: 2

70. The antibodies or antigen binding fragments according to claim 66, wherein said one or more chelators is/are selected from the group consisting of DOTA (dodecane tetraacetic acid), DTPA (diethylene triamine pentaacetic acid), NOTA (nonane tetraacetic acid) and DFO (deferoxamine) and a variant of DTPA.

71. The antibodies or antigen binding fragments according to claim 66, wherein at least one of said one or more chelators is DPTA.

72. The antibodies or antigen binding fragments according to claim 0-70, wherein said one or more chelators is/are a variant of DTPA, such as CHX-A″-DTPA or p-SCN-Bn-CHX-A″-DTPA.

73. The antibodies or antigen binding fragments according to claim 66, comprising at least two chelators.

74. The antibodies or antigen binding fragments according to claim 73, wherein said at least two chelators are DTPA, and wherein said chelator-to-antibody ratio (CAR) is 3.

75. The antibodies or antigen binding fragments according to claim 66, wherein said chelator is bound to a radioactive isotope.

76. The antibodies or antigen binding fragments according claim 75, wherein said radioactive isotope is ¹⁷⁷Lu.

77. The antibodies or antigen binding fragments according to claim 66, wherein said antibodies or antigen binding fragments further comprise an Fc region, wherein said Fc region is not reactive or exhibits little reactivity.

78. The antibodies or antigen binding fragments according to claim 66, wherein said antibody is ¹⁷⁷Lu-DTPA-8H9 antibody CAR 3 or ¹⁷⁷Lu-DTPA-8H9 antibody CAR 3.6.

79. A method of treatment and/or diagnosis of a disease in a human subject comprising administering the antibodies or antigen binding fragments thereof according to claim 75, or a pharmaceutical composition comprising the antibodies or antigen binding fragments according to claim 75, to the human subject.

80. The method according to claim 79, wherein the disease is cancer.

81. The method according to claim 80, wherein said cancer is a metastasis.

82. The method according to claim 80, wherein said cancer is prostate cancer, a desmoplastic small round cell tumor, ovarian cancer, gastric cancer, pancreatic cancer, liver cancer, renal cancer, breast cancer, non-small cell lung cancer, melanoma, alveolar rhabdomyosarcoma, embryonal rhabdomyosarcoma, Ewing sarcoma, Wilms tumor, neuroblastoma, ganglioneuroblastoma, ganglioneuroma, medulloblastoma, high-grade glioma, diffuse intrinsic pontine glioma, embryonal tumors with multilayered rosettes, or a cancer expressing B7H3.

83. The method according to claim 79, wherein said antibodies or antigen binding fragments are administered intrathecally to the subject.

84. The method according to claim 79, wherein the therapeutically effective amount is from about 10 mCi to about 200.

85. A method of manufacturing the antibodies or antigen binding fragments thereof according to claim 66, comprising the steps of:
i. providing a solution comprising said antibodies or antigen binding fragments thereof;
ii. adding a chelator to the solution, whereby the chelator reacts with said antibodies or antigen binding fragments thereof; and
iii. monitoring the reaction to obtain a desired CAR.
